(19) United States
(12) Patent Application Publication
(10) Pub. No.: US 2022/0174988 A1
(43) Pub. Date: Jun. 9, 2022

(54) AMINO ACID CHELATES FOR REDUCING OXIDATIVE STRESS

(71) Applicants: Scott L. Crain, Meade, KS (US); Robert T. Coffey, Loveland, CO (US)

(72) Inventors: Scott L. Crain, Meade, KS (US); Robert T. Coffey, Loveland, CO (US)

(21) Appl. No.: 17/605,548

(22) PCT Filed: Apr. 23, 2020
(86) PCT No.: PCT/US2020/029630

§ 371 Date: Oct. 21, 2021

Related U.S. Application Data
(60) Provisional application No. 62/837,669, filed on Apr. 23, 2019.

(51) Int. Cl. A23K 20/142; A23K 20/22; A23K 20/24; A23K 20/20; A23K 50/10

(52) U.S. Cl. CPC A23K 20/142; A23K 20/22; A23K 50/10; A23K 20/30; A23K 20/24

(57) ABSTRACT

Provided herein are formulations and methods useful in the treatment of diseases and disorders in livestock due to oxidative stress. The formulations include metal amino acid chelates.

### CROSS-REFERENCE TO RELATED APPLICATIONS

This application claims priority to U.S. Provisional Application Ser. No. 62/837,669, filed Apr. 23, 2019, which is hereby incorporated by reference in its entirety.

### BACKGROUND

Most cattle have a diet that is composed of at least some forage (grass, legumes, or silage). In fact, most beef cattle are raised on pasture from birth in the spring until autumn (7 to 9 months). Then for pasture-fed animals, grass is the forage that composes all or at least the great majority of their diet. Cattle fattened in feedlots are fed small amounts of hay supplemented with grain, grain co-products and by-products, soy and other ingredients in order to increase the energy density of the diet.

“Fed cattle” is a term for cattle pushed hard to maximize weight gain. This leads to significant abnormal metabolic toxin build up resulting from chronic oxidative stress. Fed cattle refers to animals leaving a feedlot, after fattening on a concentrated ration that are ready to be sold to a packing plant for slaughter. Beef cattle are typically sold to packers at about 1,100 to about 1400 pounds, which yields a carcass weight of about 660 to about 875 pounds.

Fed cattle and other livestock suffer from a variety of disorders and medical conditions due to the feedlot environment, diet, and feeding schedule. These disorders include chronic oxidative stress, fatigue cattle syndrome, acute interstitial pneumonia, polyarticular degeneration, osteochondrosis, hydrogen peroxide toxicity, inflammation, arthritis, lameness, and reduced mobility.

### BRIEF SUMMARY

In view of the foregoing, there is a need for compositions and methods that address the disorders and medical conditions of fed cattle and other livestock. The present disclosure addresses this need, and provides additional benefits as well.

In an aspect, provided herein are formulations for reducing oxidative stress in a mammal including one or more amino acid chelate(s) selected from zinc amino acid chelate, copper amino acid chelate, manganese amino acid chelate, magnesium amino acid chelate, or potassium amino acid complex.

In an aspect, provided herein are formulations for preventing and/or treating a lameness in a mammal comprising: one or more amino acid chelate(s) selected from one or more amino acid chelate(s) selected from zinc amino acid chelate, copper amino acid chelate, manganese amino acid chelate, magnesium amino acid chelate, and potassium amino acid complex.

In an aspect, provided herein are methods for reducing oxidative stress in a mammal including administering a formulation that includes one or more amino acid chelate(s) selected from zinc amino acid chelate, copper amino acid chelate, manganese amino acid chelate, magnesium amino acid chelate, and potassium amino acid complex.

In an aspect, provided herein are methods for preventing and/or treating cattle fatigue syndrome in a mammal, including administering to the mammal a formulation that includes one or more amino acid chelate(s) selected from zinc amino acid chelate, copper amino acid chelate, manganese amino acid chelate, magnesium amino acid chelate, and potassium amino acid complex.

In an aspect, provided herein are methods for preventing and/or treating a disease caused by oxidative stress in a mammal, including administering to the mammal a formulation that includes one or more amino acid chelate(s) selected from zinc amino acid chelate, copper amino acid chelate, manganese amino acid chelate, magnesium amino acid chelate, and potassium amino acid complex.

In an aspect, provided herein are methods for improving mobility in fed cattle, the method including administering to the cattle a formulation that includes zinc amino acid chelate, copper amino acid chelate, manganese chelate, magnesium amino acid chelate, and potassium amino acid complex daily for about 1 to about 150 days, thereby resulting in increased mobility.

In an aspect, provided herein are methods for reducing pain and/or oxidative stress in a population of animals, the method including administering to the population a formulation that includes zinc amino acid chelate, copper amino acid chelate, manganese chelate, magnesium amino acid chelate, and potassium amino acid complex, daily for about 1 to about 150 days, obtaining a sample from a subset of the animal population, and measuring in the sample one or more biomarkers of pain and/or oxidative stress. In one embodiment, reduction in the one or more biomarkers results in a reduction of pain and/or oxidative stress. In one embodiment, an initial sample is obtained from a subset of the animal population. In an embodiment, the initial sample is obtained before administration of the formulation to the population. In an embodiment, the initial sample is obtained after administration of the formulation to the population, e.g. after a first and/or subsequent administration.

### BRIEF DESCRIPTION OF THE DRAWINGS

FIG. 1 shows a schematic representation of cellular antioxidant defense mechanisms. Source: Hermes-Lima, M. (2004). Oxygen in biology and biochemistry: Role of free radicals.

FIG. 2 shows a schematic representation of lipid peroxidation and the antioxidant systems that control it. Source: Wikipedia, the free encyclopedia; Lipid Peroxidation.

FIG. 3 shows that cattle treated with the formulation provided herein have less TEAC (Trolox Equivalent Antioxidant Capacity) and less MDA (malondialdehyde, i.e. lipid peroxidation) than the non-treated group.

FIG. 4 shows that cattle treated with the formulation provided herein have less superoxide dismutase (SOD) activity than the non-treated group. The SOD activity is accelerated in the non-treated group because of the greater amount of ROS. If that activity remained elevated into the finishing phase, chronic OS would become an issue.

FIG. 5 shows the feedyard consumption pattern for total megacalories.

FIGS. 6A-6B present the anatomy of the healthy or “normal” bovine forelimb. FIG. 6A show sagittal sections of the normal right bovine forelimb carpus to the foot and the related body weight distribution. FIG. 6B show a picture of a dissected normal knee joint showing no innervation or direct blood supply, and a drawing of a normal knee joint in sagittal section.

FIGS. 7A-7B present the changes in conformation of the bovine forelimb resulting in characteristic “splay legged” stand due to growth plate inflammation and joint lesions on the medial aspect, as the animal is trying to reduce pressure on the medial joint surface. FIG. 7A shows a picture and a drawing of the “splay lagged” stance. FIG. 7B shows a picture of a dissected knee joint with a large joint erosion penetrating to bone, and a drawing of a knee joint showing erosion in sagittal section.

FIG. 8 shows the reduction in metabolic toxins over the feedyard consumption pattern. The top line shows the oxidative stress index (OSI) of animals without intervention. The lower line shows the OSI of animals given the DuoPort formulation.

FIG. 9 shows infrared imaging of reduced range of motion in front limbs. Range of motion audits were performed over a time period of 4 months. 102 pens of treated animals, totaling 8,874 heads of cattle, were tested and 43.3% of treated animals showed reduced range of motion. 98 pens of untreated animals, totaling 8,918 heads of cattle, were tested and 68.2% of untreated animals showed reduced range of motion.

FIG. 10 shows a sample growth plate and joint from audits performed. Thirty-two (32) heads of cattle were measured, and legs were sourced from animals 4-200 days on feed. Growth plate angle histology showed growth plate disorganization. Joint and leg angles were also measured. Tendon abnormalities were observed at the point of shoulder (“rotator cuff”), elbow (“tennis elbow”) and digital flexors.

FIG. 11 shows percentage breakdown of humerus elbow joints with scores ranging from 0-3; 24% of humerus elbow joints had a score of 0; 2% had a score of 0.5; 18% had a score of 1; 21% had a score of 2; and 35% had a score of 3.

FIG. 12 shows percentage breakdown of R—O elbow joints with scores ranging from 0-3. 2% of R—O elbow joints had a score of 0, 15% had a score of 0.5, 39% had a score of 1; 35% had a score of 2; and 9% had a score of 3.

FIG. 13 shows data demonstrating that Prostaglandin E-2 (PGE-2) is higher in harvest ready cattle not treated with the formulation described herein.

FIG. 14 shows a schematic representation of sample preparation. The left flow chart shows preparation of a hydrolyzed sample “3A.” The right flow chart shows preparation of a deproteinized sample “3B.”

FIG. 15 shows results of ELISA assays for 8-iso-Prostaglandin F2α (15-F2t-isoprostane), a biomarker associated with oxidative stress

FIG. 16 shows a schematic representation of the glutathione production pathway.

### DETAILED DESCRIPTION

### Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the term “control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples). In some embodiments, a control is an animal or population that has not been exposed to a compound and/or condition.

As used herein, the term “fed cattle” refers to animals leaving a feedlot after fattening on a concentrated ration, that are ready to be sold to a packing plant for slaughter.

As used herein, the term “growing phase” of fed cattle can extend from the time the animal is weaned until such time that the animal is placed on a high energy diet to enter the “finishing phase”. The “growing phase” may include a “back-grounding phase” where there is slow growth on a lower energy diet, usually <1-3 pounds of weight gain per day. The “growing phase” may include a growth phase where the animal gains <2-4 pounds per day on a moderate energy containing diet prior to the “finishing phase”. The “finishing phase” usually references the last 45-90 days that an animal is on a high energy diet to finish the weight gain process up until harvest. There are no particular time limits for any of these phases. In total, all phases can be considered the “production phase”.

As used herein, the term “beta-adrenergic agonist” refers to agonists of the beta group of adreonoreceptors. Beta adrenergic receptor agonists may be used in diets of feedlot cattle to stimulate muscle accretion. Beta agonists are non-steroidal, and they stimulate muscle accretion by increasing protein synthesis and decreasing protein catabolism. Examples of beta adrenergic agonists include ractopamine hydrochloride and zilpaterol. Ractopamine may be administered to cattle during the final 28 to 42 days before slaughter.

As used herein, the term “ionophore antibiotics” refers to compounds produced by microorganisms (mainly spore-forming bacteria) that act by specifically increasing the ion permeability of the cell membrane. Examples include nonensin and lasalocid. Ionophore antibiotics are used for control of coccidiosis and for improving feed efficiency.

As used herein, the term “oxidative stress” refers to an imbalance between the systemic manifestation of reactive oxygen species and a biological system's ability to readily detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by reactive oxygen species (ROS) generated, e.g. O₂⁻ (superoxide radical), OH (hydroxyl radical) and H₂O₂ (hydrogen peroxide). Further, some reactive oxidative species act as cellular messengers in redox signaling. Thus, oxidative stress can cause disruptions in normal mechanisms of cellular signaling and lead to a variety of diseases and disorders.

As used herein, the term “disease” or “condition” refer to a state of being or health status of an animal or subject capable of being treated with the compounds or methods provided herein. The disease may be a cancer. The disease may be an autoimmune disease. The disease may be an inflammatory disease. The disease may be a degenerative disease.

As used herein, the term “inflammatory disease” refers to a disease or condition characterized by aberrant inflammation (e.g. an increased level of inflammation compared to a control such as a healthy animal not suffering from a disease).

As used herein, the term “autoimmune disease” refers to a disease or condition in which a subject's immune system has an aberrant immune response against a substance that does not normally elicit an immune response in a healthy subject.

As used herein, the term “fatigue syndrome” refers to a physiological condition that affects livestock movement just prior to slaughter. “Cattle fatigue syndrome” or “FCS” or “fatigued cattle syndrome” refers to the condition in cattle. FCS cattle exhibit labored breathing with an abdominal grunt, lameness, muscle tremors and a reluctance to move. “Fatigued pig syndrome” refers to the condition in pigs. Some research shows it is livestock size and handling that put animals at risk for fatigue syndrome.

As used herein, the term “arthropathy” refers to any disease affecting a joint. An arthropathy can affect a single joint (monoarticular arthropathy or monoarthropathy) or many joints (polyarticular arthropathy or polyarthropathy).

As used herein, the term “degenerative joint disease” refers to damage that arises from normal stresses placed on abnormal cartilage or from abnormal stresses placed on normal cartilage (such as occurs with hip dysplasia). It can also occasionally result from trauma, hemophilia, or joint denervation. It is a disease that typically manifests clinically as slowly progressive joint pain, joint stiffness, and decreased range of motion in affected joints. It can result in the thinning of joint cartilage and joint effusion, and the production of periarticular osteophytes. As such, “polyarticular degeneration” refers to damage to many joints.

As used herein, the term “osteochondrosis” is a family of orthopedic diseases of the joint that occur in children, adolescents and other rapidly growing animals, particularly pigs, horses, dogs, and broiler chickens. They are characterized by interruption of the blood supply of a bone, in particular to the epiphysis followed by localized bony necrosis, and later, regrowth of the bone. This disorder is defined as a focal disturbance of endochondral ossification and is regarded as having a multifactorial cause, so no one thing accounts for all aspects of this disease. Reactive oxygen species detrimentally affect bone, tendon, and cartilage metabolism.

As used herein, the term “tendinopathy”, also known as “tendinitis” or “tendinosis”, refers to a type of tendon disorder that results in pain, swelling, and impaired function.

As used herein, the term “polyarthritic disease” refers to any type of arthritis that involves 2 or more joints simultaneously.

As used herein, the term “NAMI mobility score” refers to the North American Meat Institute scoring system to evaluate mobility of cattle at packing plants, providing the cattle industry with a tool to benchmark and improve the welfare of finished cattle. Mobility Score 1—Normal, walks easily with no apparent lameness or change in gait. Mobility Score 2—Exhibits minor stiffness, shortness of stride or a slight limp but keeps up with normal cattle in the group. Mobility Score 3—Exhibits obvious stiffness, difficulty taking steps, an obvious limp or obvious discomfort and lags behind normal cattle walking as a group. Mobility Score 4—Extremely reluctant to move even when encouraged by a handler; described as statue-like.

As used herein, the term “hydrogen peroxide toxicity” refers to the accumulation of hydrogen peroxide in cellular systems, individual cells, or the body circulatory system due to the inability to detoxify the hydrogen peroxide to water and oxygen.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics, which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “amino acid chelate” or “metal amino acid chelate” refers to a product resulting from the reaction of a metal ion from a soluble metal salt with amino acids. It has a mole ratio with 1 mole of metal to 1-3 (preferably 2) moles of amino acid to form coordinate covalent bonds. In an embodiment, the average weight of the amino acids is approximately 150 daltons and the resulting molecular weight of the chelate must not exceed 800 daltons. When used as a commercial feed ingredient, it may be declared as a specific metal amino acid chelate.

As used herein, the term “metal amino acid complex” refers to the product resulting from complexing of a soluble salt (such as potassium or manganese) with an amino acid(s). When used as a commercial feed ingredient, it may be declared as a specific metal amino complex: i.e., potassium amino acid complex.

As used herein, the term “metal complex” refers to the product resulting from complexing a soluble metal salt with a specific amino acid. Minimum metal content may be declared on feed labels. When used as a commercial feed ingredient, it may be declared as a specific metal, specific amino complex: i.e., copper lysine; zinc lysine; ferric methionine; manganese methionine and/or zinc methionine.

As used herein, the term “amino acid side chain” refers to the functional substituent contained on amino acids. For example, an amino acid side chain may be the side chain of a naturally occurring amino acid. Naturally occurring amino acids are those encoded by the genetic code, as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is H,

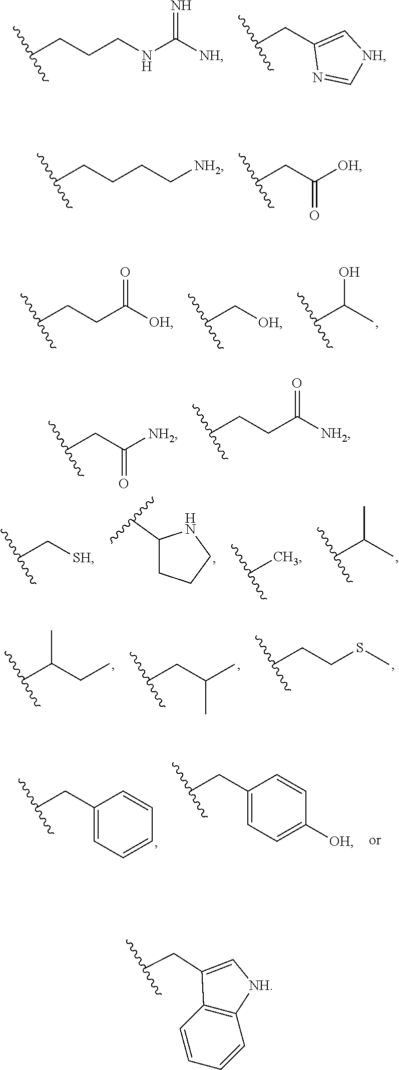

As used herein, the term “glycinate” refers to any salt or ester of glycine.

As used herein, the term “lysinate” refers to any salt or ester of lysine.

As used herein, the term “excipient” and “carrier” refer to a substance that aids the administration of an active agent to and absorption by a subject and can be included in the compositions of the present disclosure without causing a significant adverse toxicological effect on the patient. Non-limiting examples of acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “microencapsulation” is a process in which tiny particles or droplets are surrounded by a coating to give small capsules, of many useful properties. In general, it is used to incorporate food ingredients, enzymes, cells or other materials on a micro metric scale. Microencapsulation utilized herein may be useful in protecting the formulations described herein from enzymes and low pH of the rumen.

### Compositions

In an aspect, provided herein are formulations for reducing oxidative stress in a mammal including one or more components selected from zinc amino acid chelate, copper amino acid chelate, manganese amino acid chelate, magnesium amino acid chelate, or potassium amino acid complex.

In an aspect, provided herein are formulations for preventing and/or treating lameness in a mammal comprising: one or more components selected from zinc amino acid chelate, copper amino acid chelate, manganese amino acid chelate, magnesium amino acid chelate, and potassium amino acid complex.

In embodiments, the formulation comprises two or more amino acid chelate(s) selected from zinc amino acid chelate, copper amino acid chelate, manganese amino acid chelate, magnesium amino acid chelate, and potassium amino acid complex. In embodiments, the formulation comprises three or more amino acid chelate(s) selected from zinc amino acid chelate, copper amino acid chelate, manganese amino acid chelate, magnesium amino acid chelate, and potassium amino acid complex. In embodiments, the formulation comprises four or more amino acid chelate(s) selected from zinc amino acid chelate, copper amino acid chelate, manganese amino acid chelate, magnesium amino acid chelate, and potassium amino acid complex. In embodiments, the formulation comprises zinc amino acid chelate, copper amino acid chelate, manganese amino acid chelate, magnesium amino acid chelate, and potassium amino acid complex. In embodiments, the formulation includes one or more components selected from zinc amino acid chelate, copper amino acid chelate, manganese amino acid chelate, magnesium amino acid chelate, and potassium amino acid complex. In embodiments, the formulation excludes one or more components selected from zinc amino acid chelate, copper amino acid chelate, manganese amino acid chelate, magnesium amino acid chelate, and potassium amino acid complex.

In embodiments, the zinc amino acid chelate has the structure:

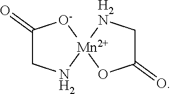

In embodiments, the zinc amino acid chelate is in the range of about 1% to about 30% (w/w) in the formulation. In embodiments, the zinc amino acid chelate is in the range of about 5% to about 15% (w/w) in the formulation. In embodiments, the zinc amino acid chelate is in the range of about 2% to about 29% (w/w) in the formulation. In embodiments, the zinc amino acid chelate is in the range of about 3% to about 28% (w/w) in the formulation. In embodiments, the zinc amino acid chelate is in the range of about 4% to about 27% (w/w) in the formulation. In embodiments, the zinc amino acid chelate is in the range of about 5% to about 26% (w/w) in the formulation. In embodiments, the zinc amino acid chelate is in the range of about 6% to about 25% (w/w) in the formulation. In embodiments, the zinc amino acid chelate is in the range of about 7% to about 24% (w/w) in the formulation. In embodiments, the zinc amino acid chelate is in the range of about 8% to about 23% (w/w) in the formulation. In embodiments, the zinc amino acid chelate is in the range of about 9% to about 22% (w/w) in the formulation. In embodiments, the zinc amino acid chelate is in the range of about 10% to about 21% (w/w) in the formulation. In embodiments, the zinc amino acid chelate is in the range of about 11% to about 20% (w/w) in the formulation. In embodiments, the zinc amino acid chelate is in the range of about 12% to about 19% (w/w) in the formulation. In embodiments, the zinc amino acid chelate is in the range of about 13% to about 18% (w/w) in the formulation. In embodiments, the zinc amino acid chelate is in the range of about 14% to about 17% (w/w) in the formulation. In embodiments, the zinc amino acid chelate is in the range of about 15% to about 16% (w/w) in the formulation. In embodiments, the zinc amino acid chelate is about 1% (w/w) in the formulation. In embodiments, the zinc amino acid chelate is about 2% (w/w) in the formulation. In embodiments, the zinc amino acid chelate is about 3%. In embodiments, the zinc amino acid chelate is about 4% (w/w) in the formulation. In embodiments, the zinc amino acid chelate is about 5% (w/w) in the formulation. In embodiments, the zinc amino acid chelate is about 6% (w/w) in the formulation. In embodiments, the zinc amino acid chelate is about 7% (w/w) in the formulation. In embodiments, the zinc amino acid chelate is about 8% (w/w) in the formulation. In embodiments, the zinc amino acid chelate is about 9% (w/w) in the formulation. In embodiments, the zinc amino acid chelate is about 10% (w/w) in the formulation. In embodiments, the zinc amino acid chelate is about 11% (w/w) in the formulation. In embodiments, the zinc amino acid chelate is about 12% (w/w) in the formulation. In embodiments, the zinc amino acid chelate is about 13% (w/w) in the formulation. In embodiments, the zinc amino acid chelate is about 14% (w/w) in the formulation. In embodiments, the zinc amino acid chelate is about 15% (w/w) in the formulation. In embodiments, the zinc amino acid chelate is about 16% (w/w) in the formulation. In embodiments, the zinc amino acid chelate is about 17% (w/w) in the formulation. In embodiments, the zinc amino acid chelate is about 18% (w/w) in the formulation. In embodiments, the zinc amino acid chelate is about 19% (w/w) in the formulation. In embodiments, the zinc amino acid chelate is about 20% (w/w) in the formulation. In embodiments, the zinc amino acid chelate is about 21% (w/w) in the formulation. In embodiments, the zinc amino acid chelate is about 22% (w/w) in the formulation. In embodiments, the zinc amino acid chelate is about 23% (w/w) in the formulation. In embodiments, the zinc amino acid chelate is about 24% (w/w) in the formulation. In embodiments, the zinc amino acid chelate is about 25% (w/w) in the formulation. In embodiments, the zinc amino acid chelate is about 26% (w/w) in the formulation. In embodiments, the zinc amino acid chelate is about 27% (w/w) in the formulation. In embodiments, the zinc amino acid chelate is about 28% (w/w) in the formulation. In embodiments, the zinc amino acid chelate is about 29% (w/w) in the formulation. In embodiments, the zinc amino acid chelate is about 30% (w/w) in the formulation.

In embodiments, the copper amino acid chelate has the structure:

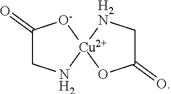

In embodiments, the copper amino acid chelate is in the range of about 1% to about 30% (w/w) in the formulation. In embodiments, the copper amino acid chelate is in the range of about 5% to about 15% (w/w) in the formulation. In embodiments, the copper amino acid chelate is in the range of about 2% to about 29% (w/w) in the formulation. In embodiments, the copper amino acid chelate is in the range of about 3% to about 28% (w/w) in the formulation. In embodiments, the copper amino acid chelate is in the range of about 4% to about 27% (w/w) in the formulation. In embodiments, the copper amino acid chelate is in the range of about 5% to about 26% (w/w) in the formulation. In embodiments, the copper amino acid chelate is in the range of about 6% to about 25% (w/w) in the formulation. In embodiments, the copper amino acid chelate is in the range of about 7% to about 24% (w/w) in the formulation. In embodiments, the copper amino acid chelate is in the range of about 8% to about 23% (w/w) in the formulation. In embodiments, the copper amino acid chelate is in the range of about 9% to about 22% (w/w) in the formulation. In embodiments, the copper amino acid chelate is in the range of about 10% to about 21% (w/w) in the formulation. In embodiments, the copper amino acid chelate is in the range of about 11% to about 20% (w/w) in the formulation. In embodiments, the copper amino acid chelate is in the range of about 12% to about 19% (w/w) in the formulation. In embodiments, the copper amino acid chelate is in the range of about 13% to about 18% (w/w) in the formulation. (w/w) in the formulation. In embodiments, the copper amino acid chelate is in the range of about 14% to about 17% (w/w) in the formulation. In embodiments, the copper amino acid chelate is in the range of about 15% to about 16% (w/w) in the formulation. In embodiments, the copper amino acid chelate is about 1% (w/w) in the formulation. In embodiments, the copper amino acid chelate is about 2% (w/w) in the formulation. In embodiments, the copper amino acid chelate is about 3% (w/w) in the formulation. In embodiments, the copper amino acid chelate is about 4% (w/w) in the formulation. In embodiments, the copper amino acid chelate is about 5% (w/w) in the formulation. In embodiments, the copper amino acid chelate is about 6% (w/w) in the formulation. In embodiments, the copper amino acid chelate is about 7% (w/w) in the formulation. In embodiments, the copper amino acid chelate is about 8% (w/w) in the formulation. In embodiments, the copper amino acid chelate is about 9% (w/w) in the formulation. In embodiments, the copper amino acid chelate is about 10% (w/w) in the formulation. In embodiments, the copper amino acid chelate is about 11% (w/w) in the formulation. In embodiments, the copper amino acid chelate is about 12% (w/w) in the formulation. In embodiments, the copper amino acid chelate is about 13% (w/w) in the formulation. In embodiments, the copper amino acid chelate is about 14% (w/w) in the formulation. In embodiments, the copper amino acid chelate is about 15% (w/w) in the formulation. In embodiments, the copper amino acid chelate is about 16% (w/w) in the formulation. In embodiments, the copper amino acid chelate is about 17% (w/w) in the formulation. In embodiments, the copper amino acid chelate is about 18% (w/w) in the formulation. In embodiments, the copper amino acid chelate is about 19% (w/w) in the formulation. In embodiments, the copper amino acid chelate is about 20% (w/w) in the formulation. In embodiments, the copper amino acid chelate is about 21% (w/w) in the formulation. In embodiments, the copper amino acid chelate is about 22% (w/w) in the formulation. In embodiments, the copper amino acid chelate is about 23% (w/w) in the formulation. In embodiments, the copper amino acid chelate is about 24% (w/w) in the formulation. In embodiments, the copper amino acid chelate is about 25% (w/w) in the formulation. In embodiments, the copper amino acid chelate is about 26% (w/w) in the formulation. In embodiments, the copper amino acid chelate is about 27% (w/w) in the formulation. In embodiments, the copper amino acid chelate is about 28% (w/w) in the formulation. In embodiments, the copper amino acid chelate is about 29% (w/w) in the formulation. In embodiments, the copper amino acid chelate is about 30% (w/w) in the formulation.

In embodiments, the manganese amino acid chelate has the structure:

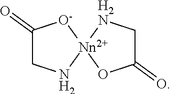

In embodiments, the manganese amino acid chelate is in the range of about 0.5% to about 25% (w/w) in the formulation. In embodiments, the manganese amino acid chelate is in the range of about 0.5% to about 10% (w/w) in the formulation. In embodiments, the manganese amino acid chelate is in the range of about 0.6% to about 24% (w/w) in the formulation. In embodiments, the manganese amino acid chelate is in the range of about 0.7% to about 23% (w/w) in the formulation. In embodiments, the manganese amino acid chelate is in the range of about 0.8% to about 22% (w/w) in the formulation. In embodiments, the manganese amino acid chelate is in the range of about 0.9% to about 21% (w/w) in the formulation. In embodiments, the manganese amino acid chelate is in the range of about 1.0% to about 20% (w/w) in the formulation. In embodiments, the manganese amino acid chelate is in the range of about 1.5% to about 19% (w/w) in the formulation. In embodiments, the manganese amino acid chelate is in the range of about 2.0% to about 18% (w/w) in the formulation. In embodiments, the manganese amino acid chelate is in the range of about 2.5% to about 17% (w/w) in the formulation. In embodiments, the manganese amino acid chelate is in the range of about 3.0% to about 16% (w/w) in the formulation. In embodiments, the manganese amino acid chelate is in the range of about 3.5% to about 15% (w/w) in the formulation. In embodiments, the manganese amino acid chelate is in the range of about 4.0% to about 14% (w/w) in the formulation. In embodiments, the manganese amino acid chelate is in the range of about 4.5% to about 13% (w/w) in the formulation. In embodiments, the manganese amino acid chelate is in the range of about 5.0% to about 12% (w/w) in the formulation. In embodiments, the manganese amino acid chelate is in the range of about 5.5% to about 11% (w/w) in the formulation. In embodiments, the manganese amino acid chelate is in the range of about 6.0% to about 10% (w/w) in the formulation. In embodiments, the manganese amino acid chelate is in the range of about 6.5% to about 9% (w/w) in the formulation. In embodiments, the manganese amino acid chelate is in the range of about 7.0% to about 8% (w/w) in the formulation. In embodiments, the manganese amino acid chelate is about 0.5% (w/w) in the formulation. In embodiments, the manganese amino acid chelate is about 0.6% (w/w) in the formulation. In embodiments, the manganese amino acid chelate is about 0.7% (w/w) in the formulation. In embodiments, the manganese amino acid chelate is about 0.8% (w/w) in the formulation. In embodiments, the manganese amino acid chelate is about 0.9% (w/w) in the formulation. In embodiments, the manganese amino acid chelate is about 1% (w/w) in the formulation. In embodiments, the manganese amino acid chelate is about 2% (w/w) in the formulation. In embodiments, the manganese amino acid chelate is about 3% (w/w) in the formulation. In embodiments, the manganese amino acid chelate is about 4% (w/w) in the formulation. In embodiments, the manganese amino acid chelate is about 5% (w/w) in the formulation. In embodiments, the manganese amino acid chelate is about 6% (w/w) in the formulation. In embodiments, the manganese amino acid chelate is about 7% (w/w) in the formulation. In embodiments, the manganese amino acid chelate is about 8% (w/w) in the formulation. In embodiments, the manganese amino acid chelate is about 9% (w/w) in the formulation. In embodiments, the manganese amino acid chelate is about 10% (w/w) in the formulation. In embodiments, the manganese amino acid chelate is about 11% (w/w) in the formulation. In embodiments, the manganese amino acid chelate is about 12% (w/w) in the formulation. In embodiments, the manganese amino acid chelate is about 13% (w/w) in the formulation. In embodiments, the manganese amino acid chelate is about 14% (w/w) in the formulation. In embodiments, the manganese amino acid chelate is about 15% (w/w) in the formulation. In embodiments, the manganese amino acid chelate is about 16% (w/w) in the formulation. In embodiments, the manganese amino acid chelate is about 17% (w/w) in the formulation. In embodiments, the manganese amino acid chelate is about 18% (w/w) in the formulation. In embodiments, the manganese amino acid chelate is about 19% (w/w) in the formulation. In embodiments, the manganese amino acid chelate is about 20% (w/w) in the formulation. In embodiments, the manganese amino acid chelate is about 21% (w/w) in the formulation. In embodiments, the manganese amino acid chelate is about 22% (w/w) in the formulation. In embodiments, the manganese amino acid chelate is about 23% (w/w) in the formulation. In embodiments, the manganese amino acid chelate is about 24% (w/w) in the formulation. In embodiments, the manganese amino acid chelate is about 25% (w/w) in the formulation.

In embodiments, the magnesium amino acid chelate has the structure:

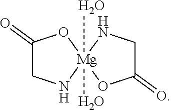

In embodiments, the magnesium amino acid chelate is in the range of about 1 to about 30% (w/w) in the formulation. In embodiments, the magnesium amino acid chelate is in the range of about 5 to about 15% (w/w) in the formulation. In embodiments, the magnesium amino acid chelate is in the range of about 2% to about 29% (w/w) in the formulation. In embodiments, the magnesium amino acid chelate is in the range of about 3% to about 28% (w/w) in the formulation. (w/w) in the formulation In embodiments, the magnesium amino acid chelate is in the range of about 5% to about 26% (w/w) in the formulation. In embodiments, the magnesium amino acid chelate is in the range of about 6% to about 25% (w/w) in the formulation. In embodiments, the magnesium amino acid chelate is in the range of about 7% to about 24% (w/w) in the formulation. In embodiments, the magnesium amino acid chelate is in the range of about 8% to about 23% (w/w) in the formulation. In embodiments, the magnesium amino acid chelate is in the range of about 9% to about 22% (w/w) in the formulation. In embodiments, the magnesium amino acid chelate is in the range of about 10% to about 21% (w/w) in the formulation. In embodiments, the magnesium amino acid chelate is in the range of about 11% to about 20% (w/w) in the formulation. In embodiments, the magnesium amino acid chelate is in the range of about 12% to about 19% (w/w) in the formulation. In embodiments, the magnesium amino acid chelate is in the range of about 13% to about 18% (w/w) in the formulation. In embodiments, the magnesium amino acid chelate is in the range of about 14% to about 17% (w/w) in the formulation. In embodiments, the magnesium amino acid chelate is in the range of about 15% to about 16% (w/w) in the formulation. In embodiments, the magnesium amino acid chelate is about 1% (w/w) in the formulation. In embodiments, the magnesium amino acid chelate is about 2% (w/w) in the formulation. In embodiments, the magnesium amino acid chelate is about 3% (w/w) in the formulation. In embodiments, the magnesium amino acid chelate is about 4% (w/w) in the formulation. In embodiments, the magnesium amino acid chelate is about 5% (w/w) in the formulation. In embodiments, the magnesium amino acid chelate is about 6% (w/w) in the formulation. In embodiments, the magnesium amino acid chelate is about 7% (w/w) in the formulation. In embodiments, the magnesium amino acid chelate is about 8% (w/w) in the formulation. In embodiments, the magnesium amino acid chelate is about 9% (w/w) in the formulation. In embodiments, the magnesium amino acid chelate is about 10% (w/w) in the formulation. In embodiments, the magnesium amino acid chelate is about 11% (w/w) in the formulation. In embodiments, the magnesium amino acid chelate is about 12% (w/w) in the formulation. In embodiments, the magnesium amino acid chelate is about 13% (w/w) in the formulation. In embodiments, the magnesium amino acid chelate is about 14% (w/w) in the formulation. In embodiments, the magnesium amino acid chelate is about 15% (w/w) in the formulation. In embodiments, the magnesium amino acid chelate is about 16% (w/w) in the formulation. In embodiments, the magnesium amino acid chelate is about 17% (w/w) in the formulation. In embodiments, the magnesium amino acid chelate is about 18% (w/w) in the formulation. In embodiments, the magnesium amino acid chelate is about 19% (w/w) in the formulation. In embodiments, the magnesium amino acid chelate is about 20% (w/w) in the formulation. In embodiments, the magnesium amino acid chelate is about 21% (w/w) in the formulation. In embodiments, the magnesium amino acid chelate is about 22% (w/w) in the formulation. In embodiments, the magnesium amino acid chelate is about 23% (w/w) in the formulation. In embodiments, the magnesium amino acid chelate is about 24% (w/w) in the formulation. In embodiments, the magnesium amino acid chelate is about 25% (w/w) in the formulation. In embodiments, the magnesium amino acid chelate is about 26% (w/w) in the formulation. In embodiments, the magnesium amino acid chelate is about 27% (w/w) in the formulation. In embodiments, the magnesium amino acid chelate is about 28% (w/w) in the formulation. In embodiments, the magnesium amino acid chelate is about 29% (w/w) in the formulation. In embodiments, the magnesium amino acid chelate is about 30% (w/w) in the formulation.

In embodiments, the potassium amino acid complex has the structure:

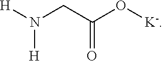

In embodiments, the potassium amino acid complex is in the range of about 1 to about 30% (w/w) in the formulation. In embodiments, the potassium amino acid complex is in the range of about 5 to about 15% (w/w) in the formulation. In embodiments, the potassium amino acid chelate is in the range of about 2% to about 29% (w/w) in the formulation. In embodiments, the potassium amino acid chelate is in the range of about 3% to about 28% (w/w) in the formulation. In embodiments, the potassium amino acid chelate is in the range of about 4% to about 27% (w/w) in the formulation. In embodiments, the potassium amino acid chelate is in the range of about 5% to about 26% (w/w) in the formulation. In embodiments, the potassium amino acid chelate is in the range of about 6% to about 25% (w/w) in the formulation. In embodiments, the potassium amino acid chelate is in the range of about 7% to about 24% (w/w) in the formulation. In embodiments, the potassium amino acid chelate is in the range of about 8% to about 23% (w/w) in the formulation. In embodiments, the potassium amino acid chelate is in the range of about 9% to about 22%. (w/w) in the formulation In embodiments, the potassium amino acid chelate is in the range of about 10% to about 21% (w/w) in the formulation. In embodiments, the potassium amino acid chelate is in the range of about 11% to about 20% (w/w) in the formulation. In embodiments, the potassium amino acid chelate is in the range of about 12% to about 19% (w/w) in the formulation. In embodiments, the potassium amino acid chelate is in the range of about 13% to about 18%. (w/w) in the formulation In embodiments, the potassium amino acid chelate is in the range of about 14% to about 17% (w/w) in the formulation. In embodiments, the potassium amino acid chelate is in the range of about 15% to about 16% (w/w) in the formulation. In embodiments, the potassium amino acid chelate is about 1% (w/w) in the formulation. In embodiments, the potassium amino acid chelate is about 2% (w/w) in the formulation. In embodiments, the potassium amino acid chelate is about 3% (w/w) in the formulation. In embodiments, the potassium amino acid chelate is about 4% (w/w) in the formulation. In embodiments, the potassium amino acid chelate is about 5% (w/w) in the formulation. In embodiments, the potassium amino acid chelate is about 6% (w/w) in the formulation. In embodiments, the potassium amino acid chelate is about 7% (w/w) in the formulation. In embodiments, the potassium amino acid chelate is about 8% (w/w) in the formulation. In embodiments, the potassium amino acid chelate is about 9% (w/w) in the formulation. In embodiments, the potassium amino acid chelate is about 10% (w/w) in the formulation. In embodiments, the potassium amino acid chelate is about 11% (w/w) in the formulation. In embodiments, the potassium amino acid chelate is about 12% (w/w) in the formulation. In embodiments, the potassium amino acid chelate is about 13% (w/w) in the formulation. In embodiments, the potassium amino acid chelate is about 14% (w/w) in the formulation. In embodiments, the potassium amino acid chelate is about 15% (w/w) in the formulation. In embodiments, the potassium amino acid chelate is about 16% (w/w) in the formulation. In embodiments, the potassium amino acid chelate is about 17% (w/w) in the formulation. In embodiments, the potassium amino acid chelate is about 18% (w/w) in the formulation. In embodiments, the potassium amino acid chelate is about 19% (w/w) in the formulation. In embodiments, the potassium amino acid chelate is about 20% (w/w) in the formulation. In embodiments, the magnesium amino acid chelate is about 21% (w/w) in the formulation. In embodiments, the potassium amino acid chelate is about 22% (w/w) in the formulation. In embodiments, the potassium amino acid chelate is about 23% (w/w) in the formulation. In embodiments, the potassium amino acid chelate is about 24% (w/w) in the formulation. In embodiments, the potassium amino acid chelate is about 25% (w/w) in the formulation. In embodiments, the potassium amino acid chelate is about 26% (w/w) in the formulation. In embodiments, the potassium amino acid chelate is about 27% (w/w) in the formulation. In embodiments, the potassium amino acid chelate is about 28% (w/w) in the formulation. In embodiments, the potassium amino acid chelate is about 29% (w/w) in the formulation. In embodiments, the potassium amino acid chelate is about 30% (w/w) in the formulation.

In embodiments, the formulation include one or more amino acid chelates as described herein. In embodiments, the formulation excludes one or more amino acid chelates as described herein.

In embodiments, the amino acid chelate is a glycinate. For example, the zinc amino acid chelate is a zinc glycinate, the copper amino acid chelate is a copper glycinate, the manganese amino acid chelate is a manganese glycinate, and/or the magnesium amino acid chelate is a magnesium glycinate.

In embodiments, the amino acid chelate is a lysinate. For example, the zinc amino acid chelate is a zinc lysinate, the copper amino acid chelate is a copper lysinate, the manganese amino acid chelate is a manganese lysinate, and/or the magnesium amino acid chelate is a magnesium lysinate.

In embodiments, the formulation further comprises one or more of N-acetyl-L-cysteine, glycine, glutamic acid, and cysteine. In embodiments, the formulation further comprises N-acetyl-L-cysteine. In embodiments, the formulation further comprises glycine. In embodiments, the formulation further comprises glutamic acid. In embodiments, the formulation further comprises cysteine. In embodiments, the formulation further comprises two or more of N-acetyl-L-cysteine, glycine, glutamic acid, and cysteine. In embodiments, In embodiments, the formulation further comprises three or more of N-acetyl-L-cysteine, glycine, glutamic acid, and cysteine. In embodiments, the formulation further comprises all four of N-acetyl-L-cysteine, glycine, glutamic acid, and cysteine. In embodiments, the formulation excludes one or more components selected from of N-acetyl-L-cysteine, glycine, glutamic acid, and cysteine.

In embodiments, the formulation further comprises N-acetyl-L-cysteine in the range of about 1% to about 10% (w/w), about 2% to about 9% (w/w), about 3% to about 8% (w/w), about 4% to about 7% (w/w), or about 5% to about 6% (w/w). In embodiments, the N-acetyl-L-cysteine is in the range of about 1% (w/w), about 2% (w/w), about 3% (w/w), about 4% (w/w), about 5% (w/w), about 6% (w/w), about 7% (w/w), about 8% (w/w), about 9% (w/w), or about 10% (w/w). In embodiments, the formulation further comprises glycine in the range of about 0.1% to about 1% (w/w), about 0.2% to about 0.9% (w/w), about 0.3% to about 0.8% (w/w), about 0.4% to about 0.7% (w/w), or about 0.5% to about 0.6% (w/w). In embodiments, the glycine is in the range of about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), or about 1% (w/w). In embodiments, the formulation further comprises glutamic acid in the range of 0.1% to about 1% (w/w), about 0.2% to about 0.9% (w/w), about 0.3% to about 0.8% (w/w), about 0.4% to about 0.7% (w/w), or about 0.5% to about 0.6% (w/w). In embodiments, the glutamic acid is in the range of about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), or about 1% (w/w). In embodiments, the formulation further comprises cysteine in the range of about 0.1% to about 1% (w/w), about 0.2% to about 0.9% (w/w), about 0.3% to about 0.8% (w/w), about 0.4% to about 0.7% (w/w), or about 0.5% to about 0.6% (w/w). In embodiments, the cysteine is in the range of about about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), or about 1% (w/w).

In embodiments, the formulation further comprises N-acetyl-L-cysteine in the range of 1 to about 1000 milligrams (mg). In embodiments, the formulation comprises about 50 to about 950 mg, about 100 to about 900 mg, about 150 to about 850 mg, about 200 to about 800 mg, about 250 to about 750 mg, about 300 to about 700 mg, about 350 to about 650 mg, about 400 to about 600 mg, about 450 to about 550 mg, or about 500 mg of N-acetyl-L-cysteine. In embodiments, the formulation comprises about 1 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 75 mg 0, about 800 mg, about 850 mg, about 900 mg, about 950 mg, or about 1000 mg of N-acetyl-L-cysteine.

In embodiments, the formulation further comprises glycine in the range of 1 to about 1000 milligrams (mg). In embodiments, the formulation comprises about 50 to about 950 mg, about 100 to about 900 mg, about 150 to about 850 mg, about 200 to about 800 mg, about 250 to about 750 mg, about 300 to about 700 mg, about 350 to about 650 mg, about 400 to about 600 mg, about 450 to about 550 mg, or about 500 mg of glycine. In embodiments, the formulation comprises about 1 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 75 mg 0, about 800 mg, about 850 mg, about 900 mg, about 950 mg, or about 1000 mg of glycine.

In embodiments, the formulation further comprises glutamic acid in the range of 1 to about 1000 milligrams (mg). In embodiments, the formulation comprises about 50 to about 950 mg, about 100 to about 900 mg, about 150 to about 850 mg, about 200 to about 800 mg, about 250 to about 750 mg, about 300 to about 700 mg, about 350 to about 650 mg, about 400 to about 600 mg, about 450 to about 550 mg, or about 500 mg of glutamic acid. In embodiments, the formulation comprises about 1 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 75 mg 0, about 800 mg, about 850 mg, about 900 mg, about 950 mg, or about 1000 mg of glutamic acid.

In embodiments, the formulation further comprises cysteine in the range of 1 to about 1000 milligrams (mg). In embodiments, the formulation comprises about 50 to about 950 mg, about 100 to about 900 mg, about 150 to about 850 mg, about 200 to about 800 mg, about 250 to about 750 mg, about 300 to about 700 mg, about 350 to about 650 mg, about 400 to about 600 mg, about 450 to about 550 mg, or about 500 mg of cysteine. In embodiments, the formulation comprises about 1 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 75 mg 0, about 800 mg, about 850 mg, about 900 mg, about 950 mg, or about 1000 mg of cysteine.

In embodiments, the formulation further comprises filler in the range of 1 to about 3000 milligrams (mg). In embodiments, the formulation comprises about 50 to about 2500 mg, about 100 to about 2000 mg, about 150 to about 1500 mg, about 200 to about 1000 mg, about 250 to about 500 mg, or about 300 to about 400 mg, of filler. In embodiments, the formulation comprises about 1 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 75 mg 0, about 800 mg, about 850 mg, about 900 mg, about 950 mg, about 1000 mg, about 1500 mg, about 2000 mg, about 2500 mg, or about 3000 mg of filler. In embodiments, the formulation comprises about 1200 mg of filler.

In embodiments, the formulation is microencapsulated. In embodiments, individual components of the formulation may be microencapsulated. In embodiments, the entire formulation is microencapsulated. Examples of methods of microencapsulation include spray drying, spray chilling, rotary disk atomization, fluid bed coating, stationary nozzle coextrusion, centrifugal head coextrusion, submerged nozzle coextrusion and pan coating.

### Methods of Use

In an aspect, provided herein are methods for reducing oxidative stress in a mammal including administering a formulation that includes one or more components selected from zinc amino acid chelate, copper amino acid chelate, manganese amino acid chelate, magnesium amino acid chelate, and potassium amino acid complex.

In an aspect, provided herein are methods for preventing and/or treating cattle fatigue syndrome in a mammal including administering a formulation that includes one or more components selected from zinc amino acid chelate, copper amino acid chelate, manganese amino acid chelate, magnesium amino acid chelate, and potassium amino acid complex.

In an aspect, provided herein are methods for preventing and/or treating a disease caused by oxidative stress in a mammal comprising administering a formulation that includes one or more components selected from zinc amino acid chelate, copper amino acid chelate, manganese amino acid chelate, magnesium amino acid chelate, and potassium amino acid complex.

In embodiments, the methods include selecting an animal that has or is susceptible to a disease caused by oxidative stress and administering a formulation that includes one or more components selected from zinc amino acid chelate, copper amino acid chelate, manganese amino acid chelate, magnesium amino acid chelate, and potassium amino acid complex.

In embodiments, the formulation comprises two or more amino acid chelate(s) selected from zinc amino acid chelate, copper amino acid chelate, manganese amino acid chelate, magnesium amino acid chelate, and potassium amino acid complex. In embodiments, the formulation comprises three or more amino acid chelate(s) selected from zinc amino acid chelate, copper amino acid chelate, manganese amino acid chelate, magnesium amino acid chelate, and potassium amino acid complex. In embodiments, the formulation comprises four or more amino acid chelate(s) selected from zinc amino acid chelate, copper amino acid chelate, manganese amino acid chelate, magnesium amino acid chelate, and potassium amino acid complex. In embodiments, the formulation comprises zinc amino acid chelate, copper amino acid chelate, manganese amino acid chelate, magnesium amino acid chelate, and potassium amino acid complex. In embodiments, the formulation excludes one or more of zinc amino acid chelate, copper amino acid chelate, manganese amino acid chelate, magnesium amino acid chelate, and potassium amino acid complex.

In an aspect, provided herein are methods for improving mobility in fed cattle, the method including administering to the cattle a formulation that includes zinc amino acid chelate, copper amino acid chelate, manganese chelate, magnesium amino acid chelate, and potassium amino acid complex daily for about 1 day to about 150 days, thereby resulting in increased mobility.

In an aspect, provided herein are methods for reducing pain and/or oxidative stress in a population of animals, the method including administering to the population a formulation that includes zinc amino acid chelate, copper amino acid chelate, manganese chelate, magnesium amino acid chelate, and potassium amino acid complex, daily for about 1 day to about 150 days, obtaining a sample from a subset of the animal population, and measuring in the sample one or more biomarkers of pain and/or oxidative stress. In embodiments, reduction in the one or more biomarkers compared to a control results in a reduction of pain and/or oxidative stress. In an embodiment, the control is an animal or population of animals not administered the formulation; the population of animals (or one of or a subset thereof) prior to administration of the formulation. In embodiments, the amount of the one or more biomarkers in the sample is compared to an amount of the biomarker(s) known to be associated with pain and/or oxidative stress, and/or compared to an amount of the biomarker(s) known to be associated with a lack of pain and/or oxidative stress.

In embodiments, the formulation is administered daily for about 1 days to 365 days. In embodiments, the formulation is administered daily for about 1 days to 300 days. In embodiments, the formulation is administered daily for about 1 days to 250 days. In embodiments, the formulation is administered daily for about 1 days to 200 days. In embodiments, the formulation is administered daily for about 1 days to 150 days. In embodiments, the formulation is administered daily for about 2 days to 149 days. In embodiments, the formulation is administered daily for about 3 to 148 days. In embodiments, the formulation is administered daily for about 4 to 147 days. In embodiments, the formulation is administered daily for about 5 to 146 days. In embodiments, the formulation is administered daily for about 6 to 145 days. In embodiments, the formulation is administered daily for about 7 to 144 days. In embodiments, the formulation is administered daily for about 8 to 143 days. In embodiments, the formulation is administered daily for about 9 to 142 days. In embodiments, the formulation is administered daily for about 10 to 141 days. In embodiments, the formulation is administered daily for about 11 to 140 days. In embodiments, the formulation is administered daily for about 12 to 139 days. In embodiments, the formulation is administered daily for about 13 to 138 days. In embodiments, the formulation is administered daily for about 14 to 137 days. In embodiments, the formulation is administered daily for about 15 to 136 days. In embodiments, the formulation is administered daily for about 16 to 135 days. In embodiments, the formulation is administered daily for about 17 to 134 days. In embodiments, the formulation is administered daily for about 18 to 133 days. In embodiments, the formulation is administered daily for about 19 to 132 days. In embodiments, the formulation is administered daily for about 20 to 131 days. In embodiments, the formulation is administered daily for about 21 to 130 days. In embodiments, the formulation is administered daily for about 22 to 129 days. In embodiments, the formulation is administered daily for about 23 to 128 days. In embodiments, the formulation is administered daily for about 24 to 127 days. In embodiments, the formulation is administered daily for about 25 to 126 days. In embodiments, the formulation is administered daily for about 26 to 125 days. In embodiments, the formulation is administered daily for about 27 to 124 days. In embodiments, the formulation is administered daily for about 28 to 123 days. In embodiments, the formulation is administered daily for about 29 to 122 days. In embodiments, the formulation is administered daily for about 30 to 121 days. In embodiments, the formulation is administered daily for about 31 to 120 days. In embodiments, the formulation is administered daily for about 32 to 119 days. In embodiments, the formulation is administered daily for about 33 to 118 days. In embodiments, the formulation is administered daily for about 34 to 117 days. In embodiments, the formulation is administered daily for about 35 to 116 days. In embodiments, the formulation is administered daily for about 36 to 115 days. In embodiments, the formulation is administered daily for about 37 to 114 days. In embodiments, the formulation is administered daily for about 38 to 113 days. In embodiments, the formulation is administered daily for about 39 to 112 days. In embodiments, the formulation is administered daily for about 40 to 111 days. In embodiments, the formulation is administered daily for about 41 to 110 days. In embodiments, the formulation is administered daily for about 42 to 109 days. In embodiments, the formulation is administered daily for about 43 to 108 days. In embodiments, the formulation is administered daily for about 44 to 107 days. In embodiments, the formulation is administered daily for about 45 to 106 days. In embodiments, the formulation is administered daily for about 46 to 105 days. In embodiments, the formulation is administered daily for about 47 to 104 days. In embodiments, the formulation is administered daily for about 48 to 103 days. In embodiments, the formulation is administered daily for about 49 to 102 days. In embodiments, the formulation is administered daily for about 50 to 101 days. In embodiments, the formulation is administered daily for about 51 to 100 days. In embodiments, the formulation is administered daily for about 52 to 99 days. In embodiments, the formulation is administered daily for about 53 to 98 days. In embodiments, the formulation is administered daily for about 54 to 97 days. In embodiments, the formulation is administered daily for about 55 to 96 days. In embodiments, the formulation is administered daily for about 56 to 95 days. In embodiments, the formulation is administered daily for about 57 to 94 days. In embodiments, the formulation is administered daily for about 58 to 93 days. In embodiments, the formulation is administered daily for about 59 to 92 days. In embodiments, the formulation is administered daily for about 60 to 91 days. In embodiments, the formulation is administered daily for about 61 to 90 days. In embodiments, the formulation is administered daily for about 62 to 89 days. In embodiments, the formulation is administered daily for about 63 to 88 days. In embodiments, the formulation is administered daily for about 64 to 87 days. In embodiments, the formulation is administered daily for about 65 to 86 days. In embodiments, the formulation is administered daily for about 66 to 85 days. In embodiments, the formulation is administered daily for about 67 to 84 days. In embodiments, the formulation is administered daily for about 68 to 83 days. In embodiments, the formulation is administered daily for about 69 to 82 days. In embodiments, the formulation is administered daily for about 70 to 81 days. In embodiments, the formulation is administered daily for about 71 to 80 days. In embodiments, the formulation is administered daily for about 72 to 79 days. In embodiments, the formulation is administered daily for about 73 to 78 days. In embodiments, the formulation is administered daily for about 74 to 77 days. In embodiments, the formulation is administered daily for about 75 to 76 days.

In embodiments, the formulation is administered starting from entry of the livestock into the growth phase. In embodiments, the formulation is administered starting from entry of the livestock into the finishing phase.

In embodiments, the disease is selected from fatigue syndrome, cattle fatigue syndrome, acute interstitial pneumonia, polyarticular degeneration, osteochondrosis, tendonopathies, polyarthritic disease, hydrogen peroxide toxicity and other disease known to be initiated or aggravated by oxidative stress and reactive oxygen species. In embodiments, the disease is fatigue syndrome. In embodiments, the disease is cattle fatigue syndrome. In embodiments, the disease is acute interstitial pneumonia. In embodiments, the disease is polyarticular degeneration. In embodiments, the disease is osteochondrosis. In embodiments, the disease is tendonopathy. In embodiments, the disease is polyarthritic disease. In embodiments, the disease is hydrogen peroxide toxicity. In embodiments, the disease is a disease known to be initiated or aggravated by oxidative stress and reactive oxygen species. In embodiments, the methods exclude a disease selected from fatigue syndrome, cattle fatigue syndrome, acute interstitial pneumonia, polyarticular degeneration, osteochondrosis, tendonopathies, polyarthritic disease, and hydrogen peroxide toxicity.

In embodiments, the mammal is a fed cattle. In embodiments, the mammal is a dairy cow. In embodiments, the mammal is a pig. In embodiments, the mammal is sheep. In embodiments, the mammal is a goat. In embodiments, the mammal is water buffalo. In embodiments, the mammal is yak. In embodiments, the mammal is bison.

In embodiments, the mammal has been previously treated with a beta-adrenergic agonist, ionophores, and/or growth or production implant, injectable or oral formulations. In embodiments, the mammal has been previously treated with a beta-adrenergic agonist. In embodiments, the mammal has been previously treated with an ionophore. In embodiments, the mammal has been previously treated with a growth or production implant, injectable or oral formulation. In embodiments, the mammal has not been previously treated with a beta-adrenergic agonist, ionophores, and/or growth or production implant, injectable or oral formulations

In embodiments, the formulation is administered at a dose of about 5 grams to about 30 grams, about 6 grams to about 29 grams, about 7 grams to about 28 grams, about 8 grams to about 27 grams, about 9 grams to about 26 grams, about 10 grams to about 25 grams, about 11 grams to about 24 grams, about 12 grams to about 23 grams, about 13 grams to about 22 grams, about 14 grams to about 21 grams, about 15 grams to about 20 grams, about 16 grams to about 19 grams, or about 17 grams to about 18 grams per animal per day. In embodiments, the formulation is administered at a dose of about 5 grams, about 6 grams, about 7 grams, about 8 grams, about 9 grams, about 10 grams, about 11 grams, about 12 grams, about 13 grams, about 14 grams, about 15 grams, about 16 grams, about 17 grams, about 18 grams, about 19 grams, about 20 grams, about 21 grams, about 22 grams, about 23 grams, about 24 grams, about 25 grams, about 26 grams, about 27 grams, about 28 grams, about 29 grams, or about 30 grams per animal per day.

In embodiments, the dose provides about 1 to about 1000 milligrams (mg) of zinc per head per day. In embodiments, the dose provides about 50 to about 950 mg, about 100 to about 900 mg, about 150 to about 850 mg, about 200 to about 800 mg, about 250 to about 750 mg, about 300 to about 700 mg, about 350 to about 650 mg, about 400 to about 600 mg, about 450 to about 550 mg, or about 500 mg of zinc per head per day. In embodiments, the dose provides about 1 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 75 mg 0, about 800 mg, about 850 mg, about 900 mg, about 950 mg, or about 1000 mg of zinc per head per day. In embodiments, the dose provides about 169.4 milligrams (mg) of zinc per head per day.

In embodiments, the dose provides about 1 to about 1000 milligrams (mg) of copper per head per day. In embodiments, the dose provides about 50 to about 950 mg, about 100 to about 900 mg, about 150 to about 850 mg, about 200 to about 800 mg, about 250 to about 750 mg, about 300 to about 700 mg, about 350 to about 650 mg, about 400 to about 600 mg, about 450 to about 550 mg, or about 500 mg of copper per head per day. In embodiments, the dose provides about 1 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 75 mg 0, about 800 mg, about 850 mg, about 900 mg, about 950 mg, or about 1000 mg of copper per head per day. In embodiments, the dose provides about 169.4 milligrams (mg) of copper per head per day.

In embodiments, the dose provides about 1 to about 1000 milligrams (mg) of manganese. per head per day. In embodiments, the dose provides about 50 to about 950 mg, about 100 to about 900 mg, about 150 to about 850 mg, about 200 to about 800 mg, about 250 to about 750 mg, about 300 to about 700 mg, about 350 to about 650 mg, about 400 to about 600 mg, about 450 to about 550 mg, or about 500 mg of manganese per head per day. In embodiments, the dose provides about 1 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 75 mg 0, about 800 mg, about 850 mg, about 900 mg, about 950 mg, or about 1000 mg of manganese per head per day. In embodiments, the dose provides about 62.5 milligrams (mg) of manganese per head per day. In embodiments, the dose provides about 70 milligrams (mg) of manganese per head per day.

In embodiments, the dose provides about 1 to about 1000 milligrams (mg) of magnesium per head per day. In embodiments, the dose provides about 50 to about 950 mg, about 100 to about 900 mg, about 150 to about 850 mg, about 200 to about 800 mg, about 250 to about 750 mg, about 300 to about 700 mg, about 350 to about 650 mg, about 400 to about 600 mg, about 450 to about 550 mg, or about 500 mg of magnesium per head per day. In embodiments, the dose provides about 1 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 75 mg 0, about 800 mg, about 850 mg, about 900 mg, about 950 mg, or about 1000 mg of magnesium per head per day. In embodiments, the dose provides about 375 milligrams (mg) of magnesium per head per day. In embodiments, the dose provides about 141.4 milligrams (mg) of magnesium per head per day.

In embodiments, the dose provides about 1 to about 1000 milligrams (mg) of potassium per head per day. In embodiments, the dose provides about 50 to about 950 mg, about 100 to about 900 mg, about 150 to about 850 mg, about 200 to about 800 mg, about 250 to about 750 mg, about 300 to about 700 mg, about 350 to about 650 mg, about 400 to about 600 mg, about 450 to about 550 mg, or about 500 mg of potassium per head per day. In embodiments, the dose provides about 1 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 75 mg 0, about 800 mg, about 850 mg, about 900 mg, about 950 mg, or about 1000 mg of potassium per head per day. In embodiments, the dose provides about 141.4 milligrams (mg) of potassium per head per day.

In embodiments, the formulation provides 150 mg of zinc, 150 mg of copper, 62.5 mg of manganese, 375 mg of magnesium, and 200 mg of potassium per head per day. In embodiments, the formulation provides 169.4 mg of zinc, 169.4 mg of copper, 70 mg of manganese, 141.4 mg of magnesium, and 141.4 mg of potassium per head per day.

In embodiments, the formulation is formulated for oral delivery. In embodiments, the formulation is a powder. In embodiments, the formulation is a liquid.

In embodiments, the disease is cattle fatigue syndrome. In embodiments, the disease is acute interstitial pneumonia. In embodiments, the disease is polyarticular degeneration. In embodiments, the disease is osteochondrosis. In embodiments, the disease is otendonopathies. In embodiments, the disease is polyarthritic disease. In embodiments, the disease is hydrogen peroxide toxicity.

In embodiments, the method further includes administering to the mammal at least one additional agent selected from a synthetic steroid, a steroid implant, or a beta-agonist (beta-adrenergic agonist). In embodiments, the method further includes administering to the mammal two additional agents selected from a synthetic steroid, a steroid implant, or a beta-agonist (beta-adrenergic agonist). In embodiments, the method further includes administering to the mammal three additional agents selected from a synthetic steroid, a steroid implant, or a beta-agonist (beta-adrenergic agonist). In embodiments, the synthetic steroid is trenbolone acetate. In embodiments, the method excludes administering an additional agent.

In embodiments, the at least one additional agent is administered before administration of the formulation. In embodiments, the at least one additional agent is administered at the same time or substantially the same time as administration of the formulation. In embodiments, the at least one additional agent is administered after administration of the formulation.

In an aspect, a method of improving mobility in cattle is provided, the method including administering to the cattle a formulation including zinc amino acid chelate, copper amino acid chelate, manganese chelate, magnesium amino acid chelate, potassium amino acid complex, and glycine the daily for about 1 day to about 150 days wherein the mobility of the cattle increases.

In embodiments, mobility is measured by NAMI mobility score and wherein an improvement in mobility is a decrease in the NAMI mobility score. In embodiments, mobility is measured by range of motion of a limb and wherein an improvement in mobility is a decrease in the reduced range of motion of a limb. In embodiments, mobility is measured by infrared imaging and wherein an improvement in mobility is a decrease in the percentage of infrared crescents. In embodiments, mobility is measured by obtaining a sample from the cattle and detecting cartilage oligomeric matrix protein in an immune assay, wherein an improvement in mobility is a decrease in the amount of cartilage oligomeric matrix protein compared to a reference value.

In embodiments, the mobility is measured by the Fed Cattle Knee joint score card system. The system scores joint lesions from 1-4, with a score of 1 indicating normal knee joint (smooth, slick surface), 2 indicating slight surface erosion, 3 indicating moderate erosion penetrating to bone, and 4 indicating large erosion (see www.grandin.com/heat.stress.lameness.html; which is incorporated herein in its entirety, and specifically as it relates to knee joint scoring).

In an aspect, a method of reducing pain and/or oxidative stress in a population of animals is provided, the method including administering to the population a formulation including zinc amino acid chelate, copper amino acid chelate, manganese chelate, magnesium amino acid chelate, and potassium amino acid complex, daily for about 1 to about 150 days, obtaining a sample from a subset of the animal population, and measuring in the sample one or more biomarkers selected from prostaglandin E-2, apolipoprotein-E, 8-isoprostane, substance P, malondialdehyde, 4-hydroxynonenal, thiobarbituric acid reactive substances, total antioxidants, glutathione peroxidase, reduced glutathione (GSH), oxidized glutathione (GSSG), haptoglobin, serum Amyloid A, 8-iso-prostaglandin F2a (8-isoprostane), protein carbonyl, hydrogen sulfide, nitric oxide or metabolites thereof, and hydrogen peroxide or metabolites, wherein reduction in the one or more biomarkers results in a reduction of pain and/or oxidative stress.

In embodiments, the method includes measuring in the sample two or more biomarkers selected from prostaglandin E-2, apolipoprotein-E, 8-isoprostane, substance P, malondialdehyde, 4-hydroxynonenal, thiobarbituric acid reactive substances, total antioxidants, glutathione peroxidase, reduced glutathione (GSH), oxidized glutathione (GSSG), haptoglobin, serum Amyloid A, 8-iso-prostaglandin F2a (8-isoprostane), protein carbonyl, hydrogen sulfide, nitric oxide or metabolites thereof, and hydrogen peroxide or metabolites thereof, wherein reduction in the two or more biomarkers results in a reduction of pain and/or oxidative stress. In embodiments, the method includes measuring in the sample three or more biomarkers selected from prostaglandin E-2, apolipoprotein-E, 8-isoprostane, substance P, malondialdehyde, 4-hydroxynonenal, thiobarbituric acid reactive substances, total antioxidants, glutathione peroxidase, reduced glutathione (GSH), oxidized glutathione (GSSG), haptoglobin, serum Amyloid A, 8-iso-prostaglandin F2a (8-isoprostane), protein carbonyl, hydrogen sulfide, nitric oxide or metabolites thereof, and hydrogen peroxide or metabolites, wherein reduction in the three or more biomarkers results in a reduction of pain and/or oxidative stress. In embodiments, the method includes measuring in the sample all six biomarkers selected from prostaglandin E-2, apolipoprotein-E, 8-isoprostane, substance P, malondialdehyde, and 4-hydroxynonenal, wherein reduction in the biomarkers results in a reduction of pain and/or oxidative stress. In embodiments, the method excludes measuring in the sample one or more biomarkers selected from prostaglandin E-2, apolipoprotein-E, 8-isoprostane, substance P, malondialdehyde, 4-hydroxynonenal, thiobarbituric acid reactive substances, total antioxidants, glutathione peroxidase, reduced glutathione (GSH), oxidized glutathione (GSSG), haptoglobin, serum Amyloid A, 8-iso-prostaglandin F2a (8-isoprostane), protein carbonyl, hydrogen sulfide, nitric oxide or metabolites thereof, and hydrogen peroxide or metabolites thereof.

In embodiments, the method includes measuring in the sample one or more biomarkers selected from prostaglandin E-2, apolipoprotein-E, 8-isoprostane, substance P, malondialdehyde, 4-hydroxynonenal, thiobarbituric acid reactive substances, total antioxidants, glutathione peroxidase, reduced glutathione (GSH), oxidized glutathione (GSSG), haptoglobin, serum Amyloid A, 8-iso-prostaglandin F2a (8-isoprostane), protein carbonyl, hydrogen sulfide, nitric oxide or metabolites thereof, and hydrogen peroxide or metabolites thereof. In embodiments, the one or more biomarker includes prostaglandin E-2. In embodiments, the one or more biomarker includes apolipoprotein-E. In embodiments, the one or more biomarker includes 8-isoprostane. In embodiments, the one or more biomarker includes substance P. In embodiments, the one or more biomarker includes malondialdehyde. In embodiments, the one or more biomarker includes 4-hydroxynonenal. In embodiments, the one or more biomarker includes thiobarbituric acid reactive substances. In embodiments, the one or more biomarker includes total antioxidants. In embodiments, the one or more biomarker includes glutathione peroxidase. In embodiments, the one or more biomarker includes reduced glutathione (GSH). In embodiments, the one or more biomarker includes oxidized glutathione (GSSG). In embodiments, the one or more biomarker includes haptoglobin. In embodiments, the one or more biomarker includes serum Amyloid A. In embodiments, the one or more biomarker includes 8-iso-prostaglandin F2a (8-isoprostane). In embodiments, the one or more biomarker includes protein carbonyl. In embodiments, the one or more biomarker includes hydrogen sulfide. In embodiments, the one or more biomarker includes nitric oxide or metabolites thereof. In embodiments, the one or more biomarker includes hydrogen peroxide or metabolites thereof. In embodiments, the method excludes measuring one or more biomarkers selected from prostaglandin E-2, apolipoprotein-E, 8-isoprostane, substance P, malondialdehyde, 4-hydroxynonenal, thiobarbituric acid reactive substances, total antioxidants, glutathione peroxidase, reduced glutathione (GSH), oxidized glutathione (GSSG), haptoglobin, serum Amyloid A, 8-iso-prostaglandin F2a (8-isoprostane), protein carbonyl, hydrogen sulfide, nitric oxide or metabolites thereof, and hydrogen peroxide or metabolites thereof.

In embodiments, the formulation is administered daily for about 1 days to 365 days. In embodiments, the formulation is administered daily for about 1 days to 300 days. In embodiments, the formulation is administered daily for about 1 days to 250 days. In embodiments, the formulation is administered daily for about 1 days to 200 days. In embodiments, the formulation is administered daily for about 1 days to 150 days. In embodiments, the formulation is administered daily for about 2 days to 149 days. In embodiments, the formulation is administered daily for about 3 to 148 days. In embodiments, the formulation is administered daily for about 4 to 147 days. In embodiments, the formulation is administered daily for about 5 to 146 days. In embodiments, the formulation is administered daily for about 6 to 145 days. In embodiments, the formulation is administered daily for about 7 to 144 days. In embodiments, the formulation is administered daily for about 8 to 143 days. In embodiments, the formulation is administered daily for about 9 to 142 days. In embodiments, the formulation is administered daily for about 10 to 141 days. In embodiments, the formulation is administered daily for about 11 to 140 days. In embodiments, the formulation is administered daily for about 12 to 139 days. In embodiments, the formulation is administered daily for about 13 to 138 days. In embodiments, the formulation is administered daily for about 14 to 137 days. In embodiments, the formulation is administered daily for about 15 to 136 days. In embodiments, the formulation is administered daily for about 16 to 135 days. In embodiments, the formulation is administered daily for about 17 to 134 days. In embodiments, the formulation is administered daily for about 18 to 133 days. In embodiments, the formulation is administered daily for about 19 to 132 days. In embodiments, the formulation is administered daily for about 20 to 131 days. In embodiments, the formulation is administered daily for about 21 to 130 days. In embodiments, the formulation is administered daily for about 22 to 129 days. In embodiments, the formulation is administered daily for about 23 to 128 days. In embodiments, the formulation is administered daily for about 24 to 127 days. In embodiments, the formulation is administered daily for about 25 to 126 days. In embodiments, the formulation is administered daily for about 26 to 125 days. In embodiments, the formulation is administered daily for about 27 to 124 days. In embodiments, the formulation is administered daily for about 28 to 123 days. In embodiments, the formulation is administered daily for about 29 to 122 days. In embodiments, the formulation is administered daily for about 30 to 121 days. In embodiments, the formulation is administered daily for about 31 to 120 days. In embodiments, the formulation is administered daily for about 32 to 119 days. In embodiments, the formulation is administered daily for about 33 to 118 days. In embodiments, the formulation is administered daily for about 34 to 117 days. In embodiments, the formulation is administered daily for about 35 to 116 days. In embodiments, the formulation is administered daily for about 36 to 115 days. In embodiments, the formulation is administered daily for about 37 to 114 days. In embodiments, the formulation is administered daily for about 38 to 113 days. In embodiments, the formulation is administered daily for about 39 to 112 days. In embodiments, the formulation is administered daily for about 40 to 111 days. In embodiments, the formulation is administered daily for about 41 to 110 days. In embodiments, the formulation is administered daily for about 42 to 109 days. In embodiments, the formulation is administered daily for about 43 to 108 days. In embodiments, the formulation is administered daily for about 44 to 107 days. In embodiments, the formulation is administered daily for about 45 to 106 days. In embodiments, the formulation is administered daily for about 46 to 105 days. In embodiments, the formulation is administered daily for about 47 to 104 days. In embodiments, the formulation is administered daily for about 48 to 103 days. In embodiments, the formulation is administered daily for about 49 to 102 days. In embodiments, the formulation is administered daily for about 50 to 101 days. In embodiments, the formulation is administered daily for about 51 to 100 days. In embodiments, the formulation is administered daily for about 52 to 99 days. In embodiments, the formulation is administered daily for about 53 to 98 days. In embodiments, the formulation is administered daily for about 54 to 97 days. In embodiments, the formulation is administered daily for about 55 to 96 days. In embodiments, the formulation is administered daily for about 56 to 95 days. In embodiments, the formulation is administered daily for about 57 to 94 days. In embodiments, the formulation is administered daily for about 58 to 93 days. In embodiments, the formulation is administered daily for about 59 to 92 days. In embodiments, the formulation is administered daily for about 60 to 91 days. In embodiments, the formulation is administered daily for about 61 to 90 days. In embodiments, the formulation is administered daily for about 62 to 89 days. In embodiments, the formulation is administered daily for about 63 to 88 days. In embodiments, the formulation is administered daily for about 64 to 87 days. In embodiments, the formulation is administered daily for about 65 to 86 days. In embodiments, the formulation is administered daily for about 66 to 85 days. In embodiments, the formulation is administered daily for about 67 to 84 days. In embodiments, the formulation is administered daily for about 68 to 83 days. In embodiments, the formulation is administered daily for about 69 to 82 days. In embodiments, the formulation is administered daily for about 70 to 81 days. In embodiments, the formulation is administered daily for about 71 to 80 days. In embodiments, the formulation is administered daily for about 72 to 79 days. In embodiments, the formulation is administered daily for about 73 to 78 days. In embodiments, the formulation is administered daily for about 74 to 77 days. In embodiments, the formulation is administered daily for about 75 to 76 days.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: The Effect of Amino Acid Chelates on Total Antioxidant Capacity, Oxidative Stress (Lipid Peroxidation), and SOD Activity in Serum of Feedlot Cattle

There are a variety of reactive oxygen species (ROS) generated in cellular aerobic metabolism creating oxidative stress (OS). These ROS include superoxide, hydroxide, and lipid hydroperoxides. If left unchecked, these ROS are damaging to cells and cellular systems leading to disease.

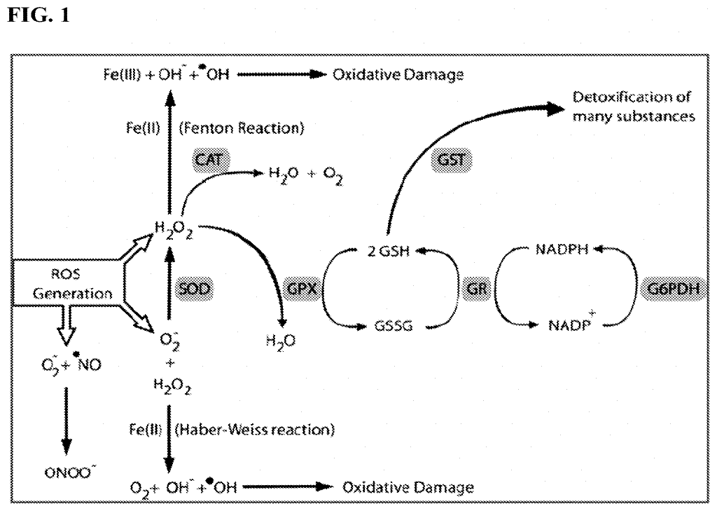

Antioxidant defense mechanisms (FIG. 1) rely on the essential trace elements Cu, Zn, Mn, and Se, and the macro elements Mg, K, and Ca for enzyme systems to function properly and for the formation of other antioxidants such as reduced glutathione. Animals undergoing oxidative stress (OS) will have a higher demand for antioxidant defense mechanisms.

The objective of this study is to demonstrate the beneficial physiological effect of two formulations (DuoPort L-30 and DuoPort e-MAX) when fed to feedlot cattle during the growing and finishing phase. Amino acid chelates with increased glycine concentration improve the metabolic state of the animal reducing the total antioxidant requirements thereby reducing oxidative stress.

Product Description & Guaranteed Analysis (DuoPort L30)

TABLE 1
Formulation of DuoPort L-30

| DuoPort ® L-30 | | | |
|---|---|---|---|
| | Label | | |
| | Guaranteed | | % of AAC |
| | Mineral Analysis | | in Formula |
| Zinc | 1.67% | Zinc AAC | 5.44% |
| Copper | 1.67% | Copper AAC | 5.55% |
| Manganese | 0.69% | Manganese AAC | 2.57% |
| Magnesium | 4.17% | Magnesium AAC | 29.55% |
| Potassium | 2.22% | Potassium | 6.43% |
| | | AA_Complex | |

AAC = Amino acid chelate

Recommended Dosage: 9 gm/head cattle/day

Recommended Days to Feed: Continuous

Product Description & Guaranteed Analysis (DuoPort e-MAX)

TABLE 2
Formulation of DuoPort e-MAX

| DuoPort ® e⁻MAX | | | |
|---|---|---|---|
| | Label | | |
| | Guaranteed | | % of AAC |
| | Mineral Analysis | | in Formula |
| Zinc | 1.58% | Zinc AAC | 5.17% |
| Copper | 1.58% | Copper AAC | 5.27% |
| Manganese | 0.66% | Manganese AAC | 2.44% |
| Magnesium | 3.64% | Magnesium AAC | 25.79% |
| Potassium | 2.05% | Potassium | 5.92% |
| | | AA_Com | |

AAC = Amino acid chelate

Recommended Dosage: 11 gm/head cattle/day

Recommended Days to Feed: Continuous

Methods

Whole blood samples were collected from all cattle at reimplant time or on the kill floor and centrifuged for 14 minutes at 3,000 RPM at 40 C to collect serum for the assays.

Three different biological assays were utilized to determine the physiological benefits of the formulation described herein and its effect on total antioxidant capacity and a reduction in oxidative stress caused by lipid hydro-peroxides and other ROS: total antioxidant assay, TBARS assay, and superoxide dismutase assay.

Total Antioxidant Assay: The assay relies on the ability of antioxidants in the sample to inhibit the oxidation of ABTS® (2,2′-azino-di-[3-ethylbenzthiazoline sulphonate]) to ABTS®⋅+ by metmyoglobin. The capacity of the antioxidants in the sample to prevent ABTS oxidation is compared with that of Trolox, a water-soluble tocopherol analogue, and is quantified as molar trolox equivalent antioxidant capacity (TEAC) and reported as mM TROLOX (Cayman Chemical Co.).

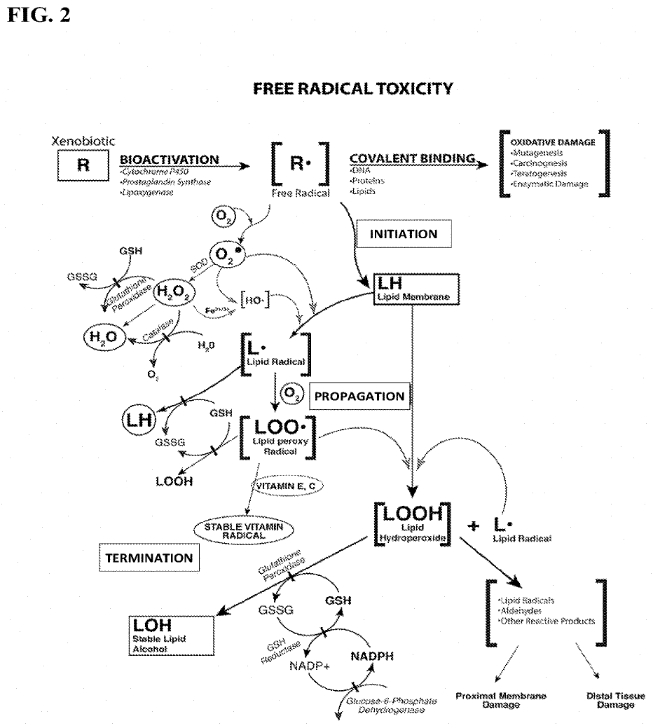

TBARS Assay: Oxidative stress (OS) in the cellular environment results in the formation of highly reactive and unstable lipid hydroperoxides. Decomposition of the unstable peroxides derived from PUFAs results in the formation of malondialdehyde (MDA), which can be quantified colorimetrically following its controlled reaction with thiobarbituric acid. The results are expressed as uM MDA and commonly referred to as the TBARS assay. A schematic of lipid peroxidation and the antioxidant systems to control it is seen in FIG. 2. The important understanding of lipid peroxidation (FIG. 2) is that the initiation begins with the superoxide, hydroxide, and/or hydroperoxyl free radical attacking the cellular lipid membrane. Adequate glutathione (GSH) and glutathione peroxidase (GPx) are necessary for the termination of lipid peroxidation. If left unchecked lipid radicals, aldehydes, and other reactive products accumulate causing tissue damage. The TBARS assay demonstrates the level of OS from Lipid Peroxidation.

Superoxide Dismutase: Significant amounts of superoxide dismutase (SOD) in cellular and extracellular environments are crucial for the prevention of diseases linked to oxidative stress. Quantification of SOD activity (Cu/Zn-SOD, Mn-SOD, Fe-SOD) is essential in order to fully characterize the antioxidant capabilities of a biological system. SOD activity is assessed by measuring the dismutation of superoxide radicals generated by xanthine oxidase and hypoxanthine. The standard curve generated using this enzyme provides a means to accurately quantify the activity of all three types of SOD (Cu/Zn—, Mn—, and Fe-SOD). Results are expressed as U/ml with a sensitivity of 0.005 U/ml.

As superoxide radicals are generated, the main function of SOD is to form hydrogen peroxide. Catalase, glutathione peroxidase, and GSH are required to terminate the reaction to water and oxygen (FIG. 1). Left unchecked, hydrogen peroxide can cause cellular damage.

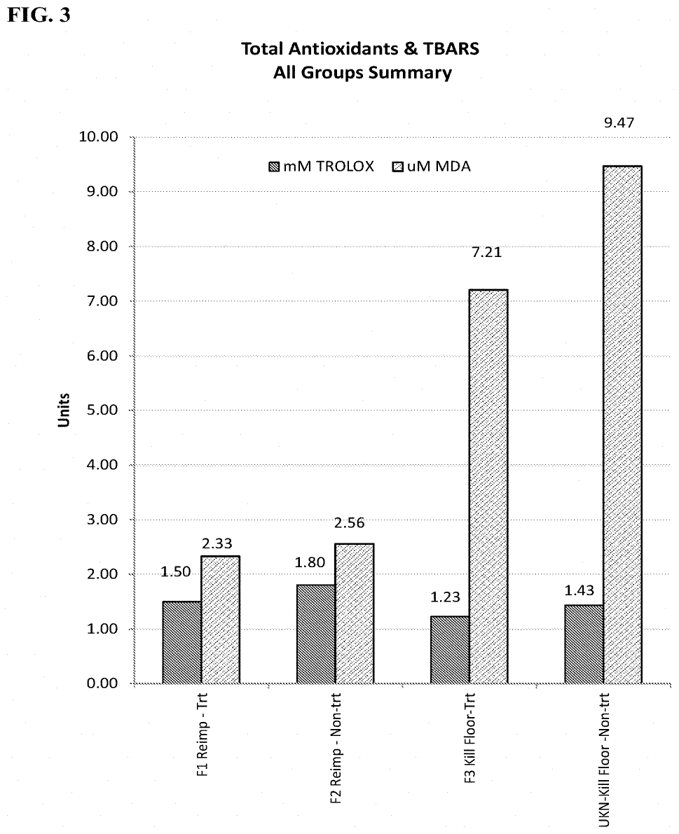

The treated ‘Reimplant Group—All Cattle’ have less TEAC and less MDA (lipid peroxidation) than the non-treated ‘Reimplant Group—All Cattle’ (see FIG. 3). The treated ‘Kill Floor Group—All Cattle’ have less TEAC and less MDA (lipid peroxidation) than the non-treated ‘Kill Floor Group—All Cattle’.

The hypothesis is that the treated cattle have less need for total antioxidant capacity because there is less ROS and oxidative stress due to the beneficial effect of the formulation described herein.

The treated ‘Kill Floor Group—All Cattle’ has maintained a higher SOD activity than the non-treated ‘Kill Floor Group—All Cattle’. It is suspected that with chronic ROS generation and sustained lipid peroxidation, i.e. chronic oxidative stress (OS), the SOD activity will decrease due to protein damage or depletion.

TABLE 3
Statistics. The significance threshold (p value) is set at .05 for this trial data. Alpha
values are .05, .01, and .001. The t-test has reliable normal distributions.

| | | | | Means | | | |
|---|---|---|---|---|---|---|---|
| | FIG | n | | DuoPort | | Statistics | |
| Group | Ref. | Trt | Non-Trt | GPX | Non-Trt | ANOVA | t-test |
| Reimplant - All Cattle | 3, 4 | | | | | | |
| mM TEAC | | 35 | 29 | 1.4997 | 1.8046 | p < .05 | p < .01 |
| uM MDA, TBARS | | 35 | 29 | 2.3312 | 2.5550 | p > .05 | p > .05 |
| U/ml SOD Activity | | 35 | 29 | 21.1197 | 36.747 | p < .001 | p < .001 |
| Reimplant - Steers | | | | | | | |
| mM TEAC | | 20 | 15 | 1.2105 | 1.6251 | p < .05 | p < .05 |
| uM MDA, TBARS | | 20 | 15 | 2.6427 | 2.6556 | p > .05 | p > .05 |
| U/ml SOD Activity | | 20 | 15 | 19.5421 | 38.7272 | p < .05 | p < .05 |
| Reimplant - Heifers | | | | | | | |
| mM TEAC | | 15 | 14 | 1.8852 | 1.9997 | p > .05 | p > .05 |
| uM MDA, TBARS | | 15 | 14 | 1.9157 | 2.4471 | p > .05¹ | P < .05¹ |
| U/ml SOD Activity | | 15 | 14 | 23.2230 | 34.7902 | p < .05 | p < .05 |
| Kill Floor - All Cattle | 3 | | | | | | |
| mM TEAC | | 61 | 60 | 1.2288 | 1.4339 | p < .05 | p < .01 |
| uM MDA, TBARS | | 61 | 60 | 7.2059 | 9.4676 | p < .01 | p < .001 |
| U/ml SOD Activity | | 61 | 60 | 13.5257 | 10.9945³ | p < .05 | p < .05 |
| Kill Floor - Steers | | | | | | | |
| mM TEAC | | 31 | 60 | 1.2204 | 1.4339 | p > .05² | P < .05² |
| uM MDA, TBARS | | 31 | 60 | 7.5053 | 9.4676 | p < .01 | p < .001 |
| U/ml SOD Activity | | 31 | 60 | 9.9071 | 10.9945 | p > .05 | p > .05 |

¹ The uM MDA for treated vs non-treated reimplant heifers had an ANOVA p < .08 which is above our significance threshold, but the t-test p < .05 indicates there is a significant difference.
² The mM TROLOX (TEAC) for treated vs non-treated kill floor steers had an ANOVA p < .07 which is above our significance threshold, but the t-test p < .05 indicates there is a significant difference.

Measuring serum physiological parameters in this initial feed trial with treated cattle vs non-treated cattle has demonstrated that, in cattle treated with the formulation described herein, there is a reduced need for circulating total antioxidants and an improvement in handling oxidative stress (OS) and reactive oxygen species (ROS). The hypothesis is validated, with data showing that amino acid chelates improve the metabolic state of the animal, reducing the total antioxidant requirements and thereby reducing oxidative stress. Simply stated, the metabolic engine runs cooler.

As demonstrated by experiments in reimplant cattle and in harvest cattle, quantified OS and ROS levels were significant on both treated and untreated cattle. Treated reimplant cattle demonstrated: 17% less antioxidant capacity requirement, 8% less lipid peroxidation per the TBARS assay, and 42% less SOD activity requirement, i.e. less oxidative stress. Treated harvest cattle demonstrated: 14% less antioxidant capacity requirement, 24% less lipid peroxidation per the TBARS assay, and 23% increase in SOD activity over non-treated harvest cattle indicative of a shutdown in SOD activity in harvest cattle due to chronic oxidative stress.

Data from these experiments show the highly biologically available chelated trace minerals are readily incorporated into antioxidant enzyme systems in the liver creating better control of the normal production of ROS in metabolic processes; the magnesium (Mg) amino acid chelate is readily available for glutathione (GSH) production in the liver; GSH is essential in the control of lipid peroxidation both intracellular and extracellular as well as the breakdown of hydrogen peroxide by glutathione peroxidase to harmless water and oxygen; subclinical potassium deficiency (hypokalemia) accelerates Mg loss through the kidneys. The potassium amino acid complex has a beneficial effect in this homeostasis.

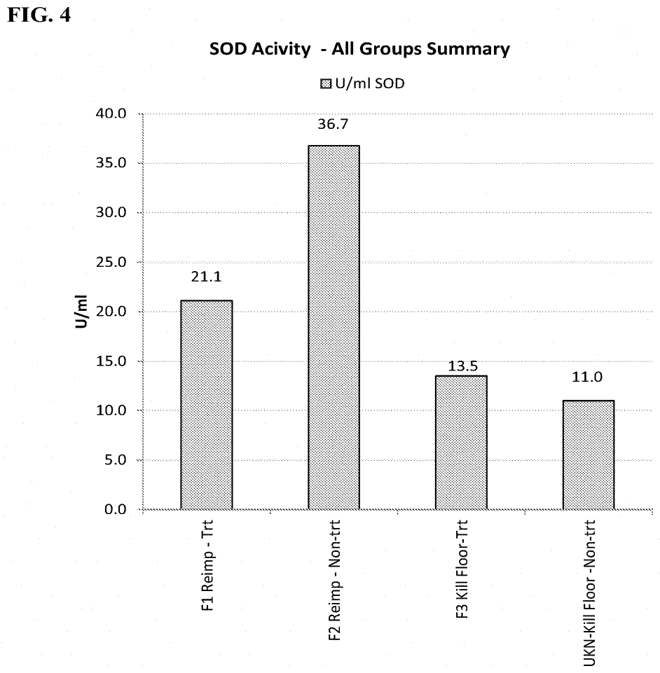

SOD Activity Shutdown in Oxidative Stress. As shown in FIG. 4, there can be a negative feedback with chronic long term OS exposure and a shutdown of SOD activity.

Kill Floor treated steers vs treated heifers have significantly different SOD activity. The explanation may be that: heifers have a higher copper requirement; heifer growth implants may have a different metabolic affect than in steers in regards to ROS production; monensin and beta-agonists (finishing phase) may have a different metabolic affect than in steers in regards to ROS production.

### Example 2: Fatigued Cattle Syndrome and Feedyard Mobility

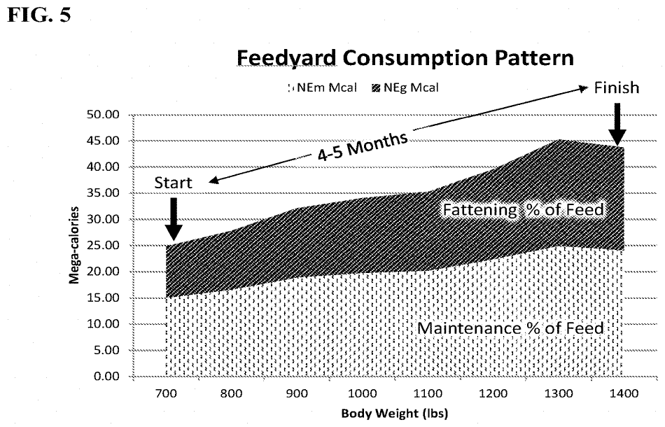

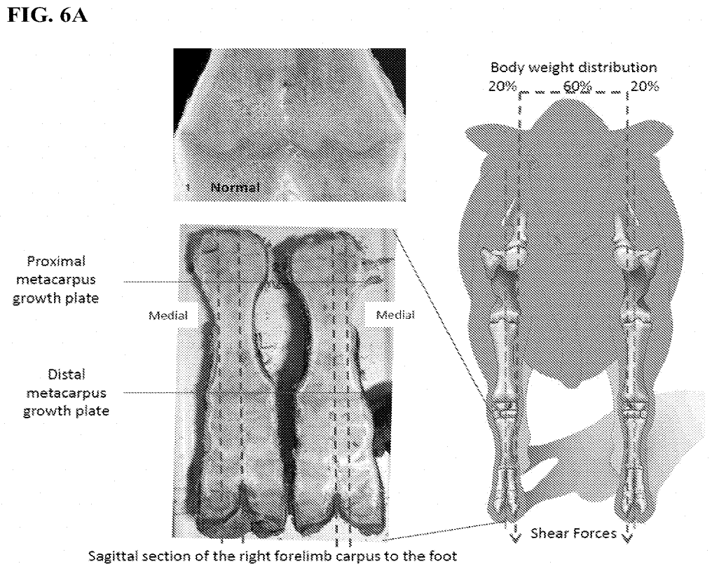

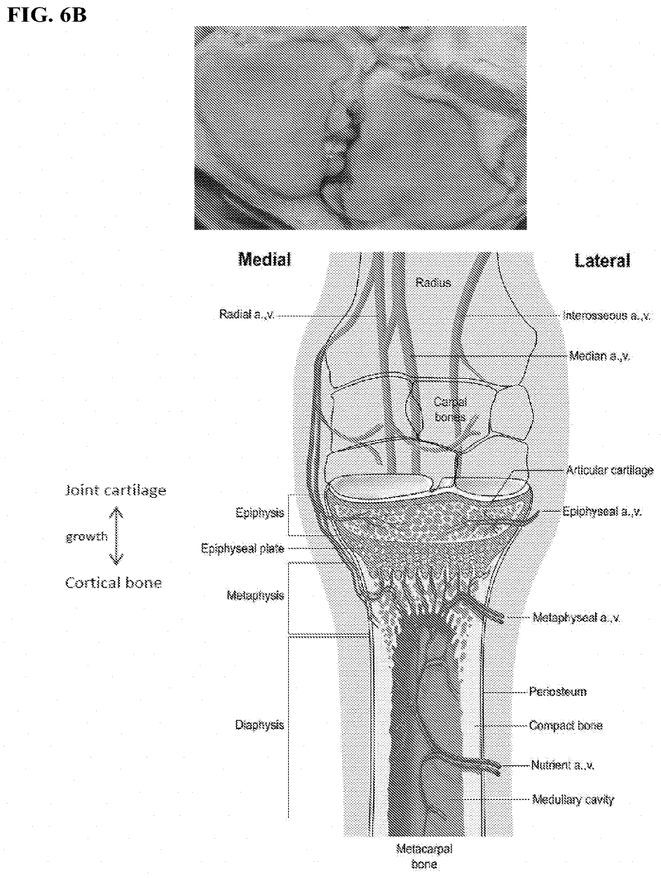

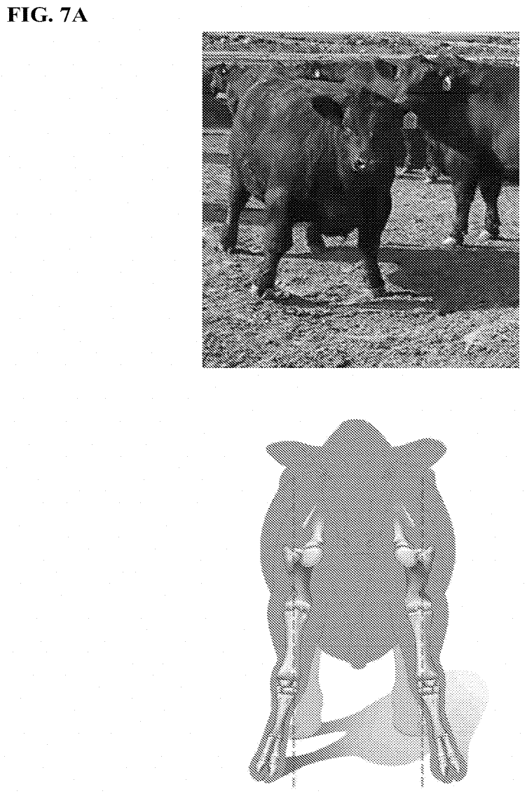

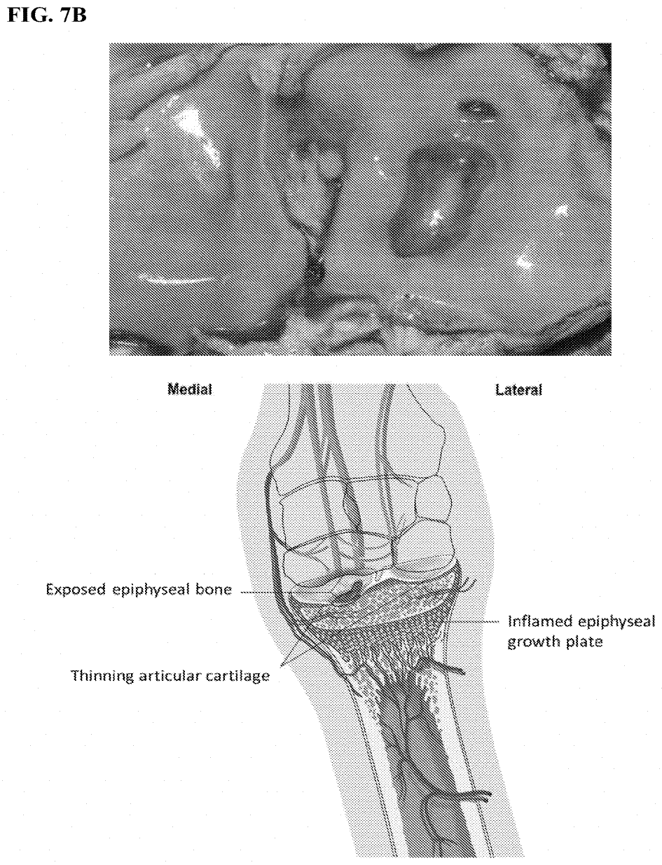

Cattle are fed for accelerated growth according to a feedyard consumption pattern (see FIG. 5). Blood assays performed over 3 years showed that more than 90% of cattle tested had elevated metabolic toxins (i.e. ROS). More than 35 international peer-reviewed studies cite that oxidative stress and endoplasmic reticulum stress damages bone, bone growth, tendons and joint surfaces. The damage to joints can be seen via infrared imaging (see FIGS. 6 and 7). Crescents visible around joints using infrared imaging are the signature of an erosion of the joint.

Fatigued Cattle Syndrome is initiated and/or propagated by production practices aimed toward rapid growth and weight gain. The pathology begins shortly after arrival at a feedyard, or may already be in progress. The pathology steadily progresses during the grow stage as cattle are brought up on feed. By the last 30 days on feed, the pathology is severe and animals are in pain.

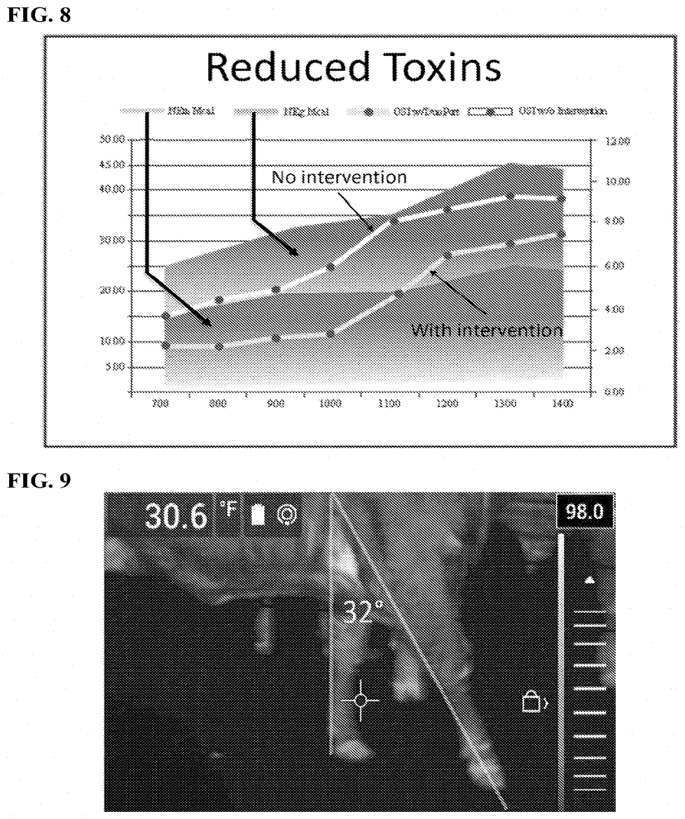

Experiments herein tested a Feedyard Fatigue Syndrome intervention formulation to address this issue. This formulation reduces levels of metabolic toxins (see FIG. 8). Audits of animals given the formulation showed that only 38.2% of treated cattle showed crescents, versus 65.5% of untreated cattle. Crescent audits were performed over a time period of 4 months. 102 pens of treated animals, totaling 8,874 heads of cattle, were tested and 38.2% of treated animals showed crescents. 98 pens of untreated animals, totaling 8,918 heads of cattle, were tested and 65.5% of untreated animals showed crescents.

The formulation also improves range of motion in treated animals. 32 degrees was considered to be a reduced range of motion for the front limb of cattle studied (see FIG. 9). Audits of animals given the formulation showed that only 43.3% of treated cattle showed a reduced range of motion, versus 68.2% of untreated cattle.

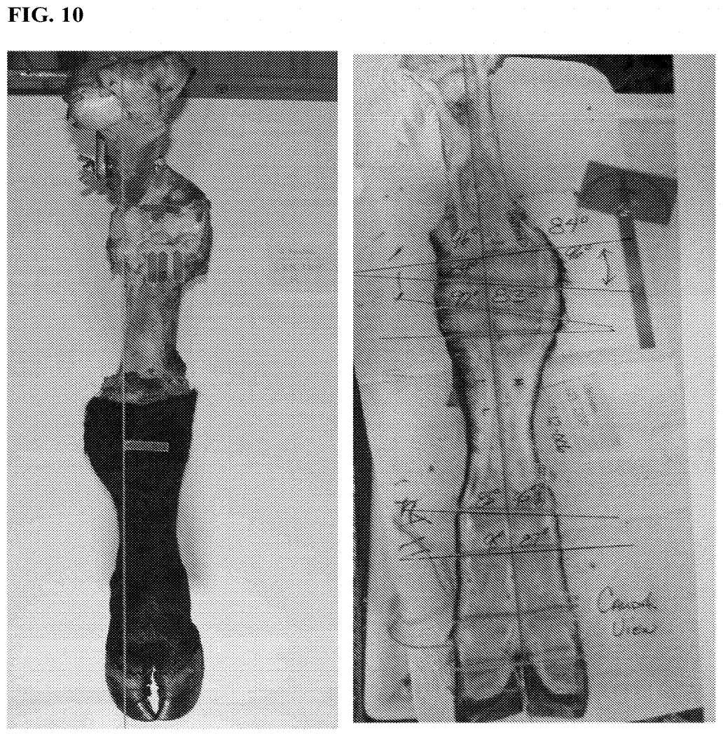

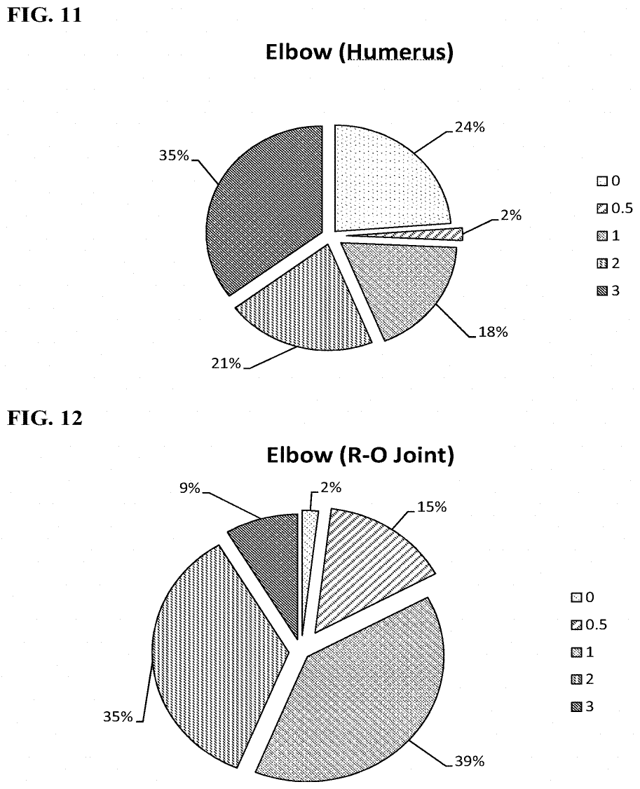

Joint stress can also be measured via feedyard necropsy audits. Audits of animals given the formulation showed that only 72% of the observed medial joint surfaces exhibited gross pathology, versus 97% in untreated animals (see FIG. 10). Feedyard necropsy audits were performed on knee joints, over a time period of 3 months. 433 treated knee joints were tested, and 72% of the observed medial joint surfaces exhibited gross pathology. 871 untreated knee joints were tested, and 97% of the observed medial joint surfaces exhibited gross pathology. Audits of joints on the harvest floor and after fabrication were also performed, to establish a preliminary baseline. Knee joint audits showed 72% and 78% of observed medial joint surfaces exhibiting gross pathology. Hock joint audits of tibial/femoral joints after fabrication in the packing plant showed 78% of medial joint surfaces exhibiting minor to severe gross pathology. Front limb audits were performed on two different elbow joints: elbow (humerus) and elbow (R—O joint). In humerus elbows, 76% had some type of lesion, and 35% had a score of 3 (see FIG. 11). In R—O joint elbows, 93% had some type of lesion, and 43% had a score of 2 or 3 (see FIG. 12).

### Example 3: Prostaglandin and Pain in Cattle

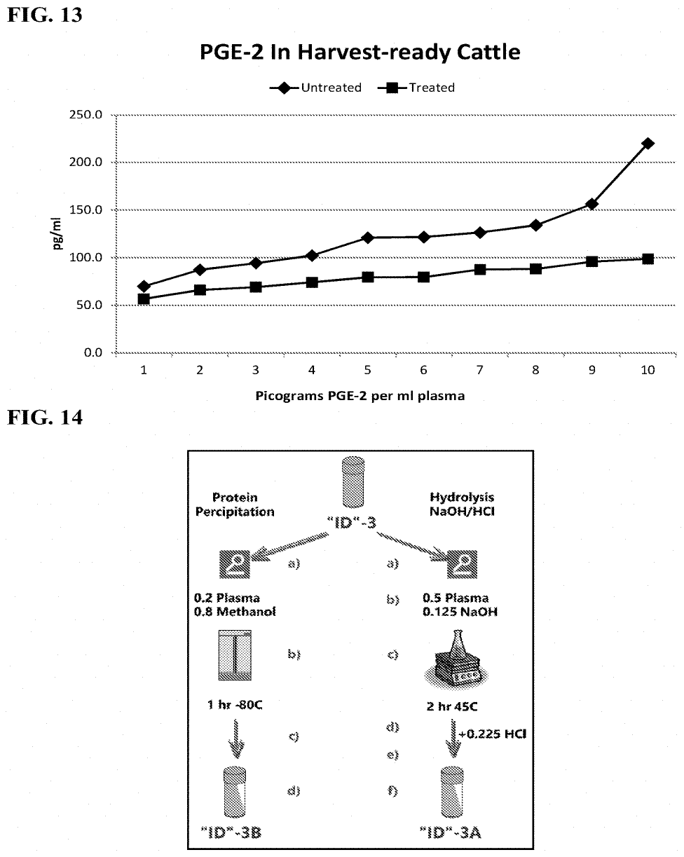

Data in the literature shows prostaglandin E-2 (PGE-2) production increases with increased oxidative stress and sensitizes the animal to peripheral pain. Consistent with this, PGE-2 is expected to be higher in untreated harvest ready animals compared to those treated with the formulations described herein (see FIG. 13).

Another biomarker that was measured was 8-iso-Prostaglandin Fat (15-F2t-isoprostane) (8-isoprostane) as described in the Methods.

Methods: Blood samples were obtained from a young Holstein Heifer from a local dairy. The heifer was manually restrained in a squeeze chute and a halter was placed to enable head restraint. Blood was drawn via the right jugular vein using Vacutainer® EDTA 10 ml blood collection tubes and a Vacutainer® tube holder with an 18-gauge Vacutainer needle. Eight blood samples were collected in series with approximately 15 seconds between each blood tube collection. Each tube was inverted at least eight times after collection for thorough mixing and then placed on ice in a cooler. The whole process of collecting eight tubes took approximately 4 minutes. The halter was removed and the heifer released from the chute and returned to her pen. Each tube contained approximately 8 ml of blood. The samples were labeled in order.

| | Sample ID | Sample ID (Plasma) |
|---|---|---|
| Tube | (Plasma) | for PMF |
| 1 | ISO1-3 | ISO-1-4 |
| 2 | ISO2-3 | ISO-2-4 |
| 3 | ISO3-3 | ISO-3-4 |
| 4 | ISO4-3 | ISO-4-4 |
| 5 | ISO5-3 | ISO-5-4 |
| 6 | ISO6-3 | ISO-6-4 |
| 7 | ISO7-3 | ISO-7-4 |
| 8 | ISO8-3 | ISO-8-4 |

Within 5 minutes after blood collection each blood tube was inoculated with 120 ul of a 2 mg/ml caffeic acid (in isotonic saline) to provide 30 ug caffeic acid per ml of whole blood. The blood tubes were inverted at least eight times after this addition. Caffeic acid acts as an antioxidant reducing lipid peroxidation in stored whole blood. The blood samples were transported on ice to our laboratory in the Research Innovation Center, CSU. Sixteen (16) 2 ml cryo-vials were properly labeled, 2 cryo-vials per blood sample, and 7.2 ul of a second antioxidant was added to each cryo-vial (4 ul/ml plasma). The purpose of this second antioxidant was to diminish lipid peroxidation in the plasma samples, i.e. platelets, Fenton reactions, etc. Within 2 hours after collection, blood samples were centrifuged at 1600 g, 4° C., 15 minutes to collect plasma aliquots. There was no visual hemolysis in any of the samples; 1.8 ml of plasma was transferred to each corresponding cryo-vial and vortexed. The cryo-vials were then stored in liquid nitrogen. Eight of the properly labeled cryo-vials were transported to the CSU Proteomics & Metabolomics Facility for analysis of 8-iso-Prostaglandin F2a (15-F2t-isoprostane) by LC-QQQ-MS. The remaining eight samples were used for ELISA analysis using the Cell BioLabs, Inc. OxiSelect™ 8-iso-Prostaglandin F2a Elisa kit, Catalog No. STA-337. Research Division Research Innovation Center—Colorado State University. There were two methods used for plasma preparation. A brief description condensed from the laboratory method is as follows:

| Plasma Deproteinization | Plasma Hydrolysis |
|---|---|
| From plasma sample “ID”-3, Phase 1, Step 5 | From plasma sample “ID”-3, Phase 1, Step 5 |
| a.) in a labeled SCV add 0.20 ml | a.) Put labeled snap cap vials in the warmed blue |
| plasma and 0.80 ml methanol, | micro-tube holder and transfer 0.5 ml plasma from |
| vortex. | “ID”-3 into labeled snap cap vial; |
| b.) place tube(s) in the −80° C. freezer for 1 | b.) add 0.125 ml 10N NaOH to each snap cap vial, |
| hour; | close cap, and invert to mix; |
| c.) Centrifuge at 13,800 x g, 4° C., 5 min. | c.) Incubate at 45° C. for 2 hrs; |
| d.) Transfer 0.5 ml of clear supernatant to a | d.) add 0.225 ml 6N HCl to each snap cap vial and |
| cryovial and label “ID”-3B. | mix; a precipitate will form. |
| df = 5 | e.) Centrifuge at 13,800 x g, 4° C., 5 min. |
| | f.) Transfer 0.5 ml of clear supernatant to a cryovial |
| | and label “ID”-3A. |
| | df = 1.7 |

Note:
“ID”-38 and “ID”-3A can be placed in −80° C. storage freezer for up to 6 months if not assayed at this time.
Proceed to 8-isoprostane assay procedure.

A schematic of “ID”-3A and “ID”-3B preparation can be seen in FIG. 14.

Sample “ID”-3A (hydrolyzed sample) preparation for anaylsis: The raw sample supernatant has a pH<1 and must be adjusted with the kit Neutralizing Solution. 20 μl of sample+60 μl of neutralizing solution gave a pH of 6-8. The total dilution factor was then 6.8. 55 μl of sample was used per the kit assay instructions; ABS at 450 nm.

Sample “ID”-3B (deproteinized sample) preparation for anaylsis: Transfer 0.2 ml of the supernatant to a 2 ml snap cap vial (SCV). A ThermoFisher Savant™ SPD1010 vacuum concentrator was used, 80 minutes, room temperature, to evaporate the methanol. Sample was dry. Reconstitute with 0.2 ml kit Sample Diluent and vortex vigorously for 15 sec.; pH=6.8-7.5; dilution factor is still 5. 55 μl of sample was used per the kit assay instructions; ABS at 450 nm.

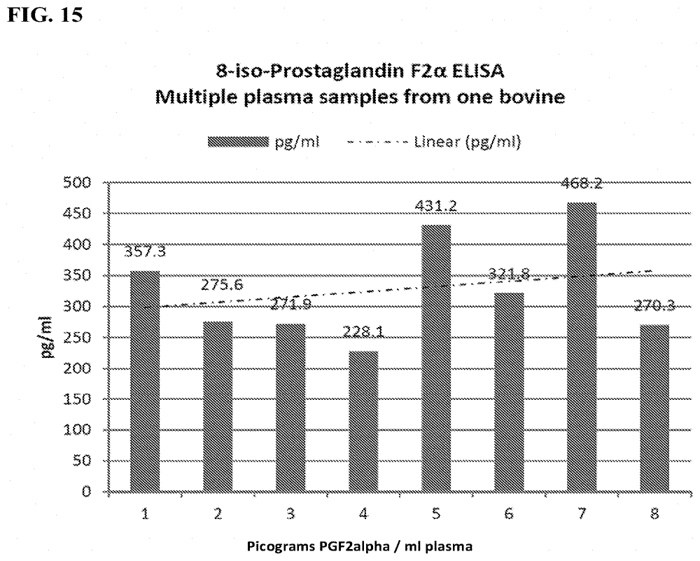

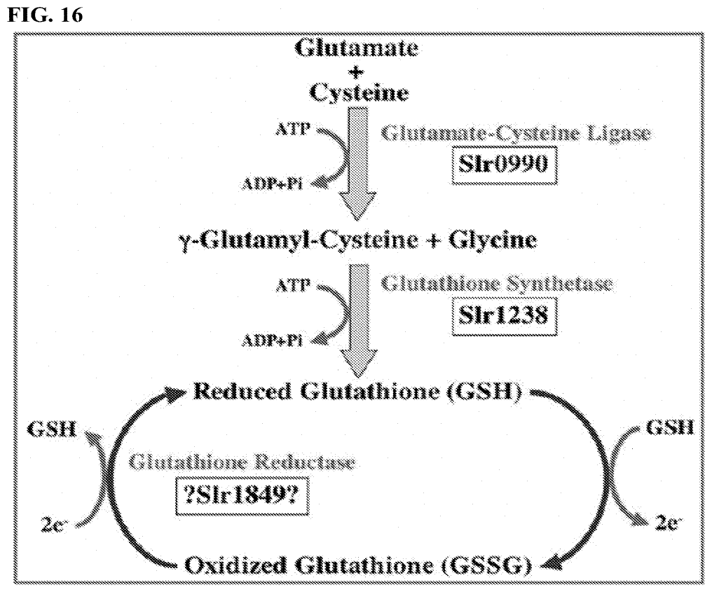

These samples were prepared for assay two separate times. Results were below the minimum standard curve detection range suggestive that this sample preparation for ELISA will not work, or the samples were <19 pg/ml, i.e. inadequate sensitivity (see FIG. 15 and Table 4).

TABLE 4
Results for sample “ID”-3A (hydrolyzed sample).

| Time(sec) | Sample | pg/ml | nM* |
|---|---|---|---|
| 0 | 1 | 357.3 | 1.27 |
| 30 | 2 | 275.6 | 0.98 |
| 60 | 3 | 271.9 | 0.97 |
| 90 | 4 | 228.1 | 0.81 |
| 120 | 5 | 431.2 | 1.54 |
| 150 | 6 | 321.8 | 1.15 |
| 180 | 7 | 468.2 | 1.67 |
| 210 | 8 | 270.3 | 0.96 |

| | | |
|---|---|---|
| Average | 328.1 | 1.17 |
| Std. Dev | 84.9 | 0.302 |

| | |
|---|---|
| Avg. CV % | 23.70% |

Std. Curve r² = 0.999 and Std. Curve Avg. CV % = 7.6%.
*nM concentrations calculated using 8-isoprostane MW = 280.54 gm/M.

### Example 4: Supplementation Reduces Oxidative Stress Index (OSI) and Improves Magnesium Status in Dairy Cattle

To establish a baseline of normal OSI levels, two clinically healthy dairy herds from the Fond du Lac, Wis. area (Herd 1 and Herd 2), certified clinically normal and healthy by the attending veterinarian, had blood samples drawn and serum collected to measure the OSI and the acute phase proteins haptoglobin and serum amyloid A. The four stages of production sampled were: Pre-fresh; Post fresh; 60-90 Days in Milk; and 250-280 Days in Milk The assay used to test for lipid peroxidation in cell membranes was the TBARS assay (thiobarbituric acid reactive substances); TBARS (TCA Method) assay from Cayman Chemical, Ann Arbor, Mich. It is a sensitive and simple assay. Malondialdehyde (MDA), formed from the peroxidation of polyunsaturated fatty acids (PUFA's), serves as a convenient index for determining the extent of peroxidation reaction. MDA reacts with 2-thiobarbituric acid (TBA) under high temperature and acidic conditions to give a red species which was measured spectrophotometrically at 530-540 nm. Serum was the tissue being analyzed. The magnesium assay was from AdipoGen Life Sciences, San Diego, Calif.; Xylidyl Blue-I Colorimetric Method.

Herd 1 was supplemented with the formulation described herein for 70-76 days before blood samples were drawn. The attending veterinarian elected to supplement the Pre-fresh and Post-fresh groups at 10 gm/hd/day, and the results indicated a very beneficial effect. The DIM 60-90 and DIM 250-280 groups were supplemented at the recommended 7 gm/hd/day. Herd 2 had serum samples collected within a week of Herd 1 to compare the supplemented Herd 1 to the non-supplemented Herd 2 which was still considered clinically healthy.

Supplementation with the formulation of the present disclosure had a statistically significant impact on the OSI and the magnesium status when supplemented for at least 60-70 days. Keep in mind that Herd 1 and Herd 2 were clinically normal herds during this trial and study. Oxidative stress was documented as more severe in the transition phase after calving. That observation was not prevalent in these normal healthy herds. The observed production benefits from supplementing are as follows: A reduction in the oxidative stress from the oxidation of NEFA and Propionate (starch) and its effect on Insulin/Insulin sensitivity and DMI during the post fresh and post peak period; Higher peak milk production and better persistency of milk post peak; A reduction of many clinical diseases, such as: Retained placenta, Metritis, Subclinical Fatty Liver disease and Ketosis post fresh, a significant reduction in mid to late lactation indigestions, a reduction in abomasal ulcers and HBS (Hemorrhagic Bowel Syndrome); Milk Quality improved with lower new infection rate and lower high fresh rate and less clinical mastitis which results in a reduction of herd somatic cell count (SCC). There are numerous variables that can influence these production benefits, but amino acid chelate provides a better metabolic environment for reducing oxidative stress during the various stages of production.

### Example 5: Prostaglandin and Pain in Cattle

Data in the literature shows prostaglandin E-2 (PGE-2) production increases with increased oxidative stress and sensitizes the animal to peripheral pain. Consistent with this, PGE-2 is expected to be higher in untreated harvest ready animals compared to those treated with the formulations described herein. Another biomarker that will be measured is 8-iso-Prostaglandin F2α (15-F2t-isoprostane) as described in the Methods.

Methods: Blood samples will be obtained as described in Example 3. The formulation will contain increased zinc and magnesium.

### Example 6: Supplementation Reduces Oxidative Stress Index (OSI) and Improves Magnesium Status in Dairy Cattle

To establish a baseline of normal OSI levels, two clinically healthy dairy herds from the Fond du Lac, Wis. area (Herd 1 and Herd 2), certified clinically normal and healthy by the attending veterinarian, will have blood samples drawn and serum collected to measure the OSI and the acute phase proteins haptoglobin and serum amyloid A as described in Example 4. The formulation will contain increased zinc and magnesium.

### REFERENCES

1) Oxidative Stress, Antioxidants, and Animal Function, Miller, J K et al, 1993 J Dairy Sci 76:2812-282.
2.) A Review: Oxidative Stress during Lactation in Dairy Cattle. Moolchandani, A., Sareen, M., Dairy and Vet Sci J 5(4): JDVS.MS.ID.555669 (2018).
3.) Oxidative stress and antioxidant status in dairy cows during prepartal and postpartal periods. Konvičná, J. et al, ACTA VET. BRNO 2015, 84: 133-140.
4.) Impact of oxidative stress on the health and immune function of dairy cattle. Veterinary Immunology and Immunopathology, Volume 128, Issues 1-3, 15 Mar. 2009, P. 104-109.
5.) Significance of insulin resistance and oxidative stress in dairy cattle with subclinical ketosis during the transition period. Youssef, M., El-Ashker, M., Tropical Animal Health and Production, February 2017, Volume 49, Issue 2, pp 239-244.
6.) The hepatic oxidation theory of the control of feed intake and its application to ruminants. M. S. Allen, B. J. Bradford, M. Oba; J. Anim. Sci. 2009.87:3317-3334.
7.) Association of oxidative status and insulin sensitivity in periparturient dairy cattle: An observational study. Abeulo, A. et al. J Anim Physiol Anim Nutr. April 2016.
8.) Oxidative Stress and Imbalance of Mineral Metabolism Contribute to Lameness in Dairy Cows. Xue-Jun Zhao et al, Biological Trace Element Research, March 2015, Volume 164, Issue 1, pp 43-49.
9.) Inflammatory Responses to Sub-Acute Ruminal Acidosis. Tanya F. Gressley, Department of Animal and Food Sciences, University of Delaware.
10.) Oxidative Stress: An Essential Factor in the Pathogenesis of Gastrointestinal Mucosal Diseases. Bhattacharyya, A., Chattopadhyay, R., Mitra, S., Crowe, S. E. (2014). Physiol Rev.; 94(2): 329-354.
11.) Catalase, Cu/Zn-Superoxide Dismutase, Glutathione Peroxidase: Their Relationship to Oxygen Utilization in Cellular Physiology, Clinical and Sub-clinical Disease, Nutrition and Trace Element Utilization in Livestock, Coffey, R. T. (1988) AABP Bovine Proceedings 23: 138-143.
12.) Applied Trace Element Nutrition in the Bovine Animal. Coffey, R. T. (1990), AABP Bovine Proceedings 22: 153-169.
13.) Glutathione synthesis. Lu, S. C. Biochim Biophys Acta. 2013 May; 1830(5): 3143-3153.
14.) Ruminal Magnesium Absorption: Mechanisms, Modulation and Meaning for Assessment of Mg Intake ISBN: 978-3-86387-651-7, Zugl: Berlin, Freie Univ., Diss., 2014, Dissertation, Freie Universitat Berlin, D 188.

What is claimed is:

1. A formulation for reducing oxidative stress in a mammal comprising: one or more components selected from zinc amino acid chelate, copper amino acid chelate, manganese amino acid chelate, magnesium amino acid chelate, and potassium amino acid complex.

2. The formulation of claim 1, wherein the formulation comprises zinc amino acid chelate, copper amino acid chelate, manganese chelate, magnesium amino acid chelate, and potassium amino acid complex.

3. The formulation of any one of claims 1-2, wherein the amino acid chelate is a glycinate.

4. The formulation of any one of the preceding claims, wherein the formulation is formulated for oral delivery.

5. The formulation of any one of the preceding claims, wherein the formulation is a powder.

6. The formulation of any one of claims 1-4, wherein the formulation is a liquid

7. The formulation of any one of the preceding claims, wherein zinc amino acid chelate is in the range of about 1% to about 30% by weight.

8. The formulation of claim 7, wherein the zinc amino acid chelate is in the range of about 5% to about 15% by weight.

9. The formulation of any one of the preceding claims, wherein copper amino acid chelate is in the range of about 1% to about 30% by weight.

10. The formulation of claim 9, wherein the copper amino acid chelate is in the range of about 5% to about 15% by weight.

11. The formulation of any one of the preceding claims, wherein manganese amino acid chelate is in the range of about 0.5% to about 25% by weight.

12. The formulation of claim 10, wherein manganese amino acid chelate is in the range of about 0.5% to about 10% by weight.

13. The formulation of any one of the preceding claims, wherein magnesium amino acid chelate is in the range of about 1% to about 30% by weight.

14. The formulation of claim 13, wherein the magnesium amino acid chelate is in the range of about 5% to about 15% by weight.

15. The formulation of any one of the preceding claims, wherein potassium amino acid complex is in the range of about 1% to about 30% by weight.

16. The formulation of claim 13, wherein potassium amino acid complex is in the range of about 5% to about 15% by weight.

17. A formulation for preventing and/or treating lameness in a mammal comprising: one or more amino acid chelate(s) selected from one or more components selected from zinc amino acid chelate, copper amino acid chelate, manganese amino acid chelate, magnesium amino acid chelate, and potassium amino acid complex.

18. The formulation of claim 17, wherein the amino acid chelate is a glycinate.

19. The formulation of any one of claim 17 or 18 wherein the formulation comprises zinc amino acid chelate, copper amino acid chelate, manganese chelate, magnesium amino acid chelate, and potassium amino acid complex.

20. The formulation of any one of claims 17-19, wherein the formulation is formulated for oral delivery.

21. The formulation of any one of claims 17-20, wherein the formulation is a powder.

22. The formulation of any one of claims 17-21, wherein zinc amino acid chelate is in the range of about 1% to about 30%.

23. The formulation of any one of claims 17-22, wherein the zinc amino acid chelate is in the range of about 5% to about 15%.

24. The formulation of any one of claims 17-23, wherein copper amino acid chelate is in the range of about 1% to about 30%.

25. The formulation of any one of claims 17-24, wherein the copper amino acid chelate is in the range of about 5% to about 15%.

26. The formulation of any one of claims 17-25, wherein manganese amino acid chelate is in the range of about 0.5% to about 25%.

27. The formulation of any one of claims 17-26, wherein manganese amino acid chelate is in the range of about 0.5% to about 10%.

28. The formulation of any one of claims 17-27, wherein magnesium amino acid chelate is in the range of about 1 to about 30%.

29. The formulation of any one of claims 17-28, wherein the magnesium amino acid chelate is in the range of about 5 to about 15%.

30. The formulation of any one of claims 15-29, wherein potassium amino acid complex is in the range of about 1 to about 30%.

32. The formulation of any one of claims 17-30, wherein potassium amino acid complex is in the range of about 5 to about 15%.

33. The formulation of any of the preceding claims, further comprising one or more of N-acetyl-L-cysteine, glycine, glutamic acid, and cysteine.

34. The formulation of claim 33 wherein the N-acetyl-L-cysteine is in the range of about 1% to about 10%, glycine in in the range of about 0.1% to about 1%, glutamic acid is in the range of 0.1% to about 1%, and/or cysteine is in the range of about 0.1% to about 1%.

35. A method for reducing oxidative stress in a mammal comprising administering a formulation comprising one or more components selected from zinc amino acid chelate, copper amino acid chelate, manganese amino acid chelate, magnesium amino acid chelate, and potassium amino acid complex.

36. A method for preventing and/or treating cattle fatigue syndrome in a mammal comprising administering a formulation comprising one or more components selected from zinc amino acid chelate, copper amino acid chelate, manganese amino acid chelate, magnesium amino acid chelate, and potassium amino acid complex.

37. A method for preventing and/or treating a disease caused by oxidative stress in a mammal comprising administering a formulation comprising one or more components selected from zinc amino acid chelate, copper amino acid chelate, manganese amino acid chelate, magnesium amino acid chelate, and potassium amino acid complex.

38. The method of claim 37, wherein the disease is selected from cattle fatigue syndrome, polyarticular degeneration, osteochondrosis, tendonopathies, polyarthritic disease, or hydrogen peroxide toxicity.

39. The method of any one of claims 35-38, wherein the mammal is a fed cattle.

40. The method of any one of claims 35-38, wherein the mammal is a dairy cow.

41. The method of any one of claims 35-38, wherein the mammal is a pig.

42. The method of any one of claims 35-41, wherein the mammal has been treated with a beta-adrenergic agonist, ionophores, and/or growth or production implant, injectable or oral formulations.

43. The method of any one of claims 35-42, wherein the formulation is administered at a dose of 7 grams per animal per day.

44. The method of any one of claims 35-43, wherein the formulation comprises zinc amino acid chelate, copper amino acid chelate, magnesium amino acid chelate and potassium amino acid complex.

45. The method of any one of claims 35-44, wherein the formulation is formulated for oral delivery.

46. The method of any one of claims 34-45, wherein the formulation is a powder.

47. The method of any one of claims 35-46, wherein zinc amino acid chelate is in the range of about 1% to about 30%.

48. The method of any one of claims 35-47, wherein the zinc amino acid chelate is in the range of about 5% to about 15%.

49. The formulation of any one of claims 35-48, wherein copper amino acid chelate is in the range of about 1 to about 30%.

50. The method of any one of claims 35-48, wherein the copper amino acid chelate is in the range of about 5% to about 15%.

51. The method of any one of claims 35-50, wherein manganese amino acid chelate is in the range of about 0.5% to about 25%.

52. The method of any one of claims 35-51, wherein manganese amino acid chelate is in the range of about 0.5% to about 10%.

53. The method of any one of claims 35-52, wherein magnesium amino acid chelate is in the range of about 1% to about 30%.

54. The method of any one of claims 35-53, wherein the magnesium amino acid chelate is in the range of about 5% to about 15%.

55. The method of any one of claims 35-54, wherein potassium amino acid complex is in the range of about 1% to about 30%.

56. The method of any one of claims 35-55, wherein potassium amino acid complex is in the range of about 5% to about 15%.

57. The method of any one of claims 35-56, further comprising administering to the mammal at least one additional agent selected from a synthetic steroid, a steroid implant, or a beta-agonist (beta-adrenergic agonist).

58. The method of claim 57, wherein the synthetic steroid is trenbolone acetate.

59. The method of claim 57 or 58, wherein the at least one additional agent is administered before administration of the formulation.

60. The method of claim 57 or 58, wherein the at least one additional agent is administered at the same time or substantially the same time as administration of the formulation.

61. The method of claim 57 or 58, wherein the at least one additional agent is administered after administration of the formulation.

62. A method for improving mobility in fed cattle, the method comprising administering to the cattle a formulation comprising zinc amino acid chelate, copper amino acid chelate, manganese chelate, magnesium amino acid chelate, and potassium amino acid complex, daily for about 1 to about 150 days thereby resulting in increased mobility.

63. The method of claim 62, wherein the amino acid chelate is a glycinate.

64. The method of claim 62 or 63, wherein mobility is measured by NAMI mobility score and wherein an improvement in mobility is a decrease in the NAMI mobility score

65. The method of claim 62 or 63, wherein mobility is measured by range of motion of a limb and wherein an improvement in mobility is a decrease in the reduced range of motion of a limb.

66. The method of claim 62 or 63, wherein mobility is measured by infrared imaging and wherein an improvement in mobility is a decrease in the percentage of infrared crescents.

67. The method of claim 62 or 63, wherein mobility is measured by obtaining a sample from the cattle and detecting cartilage oligomeric matrix protein in an immune assay, wherein an improvement in mobility is a decrease in the amount of cartilage oligomeric matrix protein compared to a reference value.

68. A method for reducing pain and/or oxidative stress in a population of animals, the method comprising:
(i) administering to the population a formulation comprising zinc amino acid chelate, copper amino acid chelate, manganese chelate, magnesium amino acid chelate, and potassium amino acid complex, daily for about 1 to about 150 days;
(ii) obtaining a sample from a subset of the animal population; and
(iii) measuring in the sample one or more biomarkers of pain and/or oxidative stress wherein reduction in the one or more biomarkers results in a reduction of pain and/or oxidative stress.

69. The method of claim 68, wherein the biomarker is selected from prostaglandin E-2, apolipoprotein-E, 8-isoprostane, substance P, malondialdehyde, 4-hydroxynonenal, thiobarbituric acid reactive substances, total antioxidants, glutathione peroxidase, reduced glutathione (GSH), oxidized glutathione (GSSG), haptoglobin, serum Amyloid A, 8-iso-prostaglandin F2α (8-isoprostane), protein carbonyl, hydrogen sulfide, nitric oxide or metabolites thereof, and hydrogen peroxide or metabolites thereof.
